SEQUENCE LISTING (3 sequences)

SEQ ID NO: 1 - DNA, 19 bp, Artificial Sequence
  Synthetic HT1D mRNA flanjkin primer
cccggagtcgaatcctgaa

SEQ ID NO: 2 - DNA, 22 bp, Artificial Sequence
  Synthetic HT1D mRNA flanking primer
tgataagctgtgctgtggtgaa

SEQ ID NO: 3 - DNA, 23 bp, Artificial Sequence
  Synthetic probe
  modified_base (1)..(1): labelled with 6-carboxy fluorescein-aminohexylamidite
  modified_base (23)..(23): labelled with black hole quencher
ctatcttggtcatgcccatcagc

(19) United States
(12) Patent Application Publication
(10) Pub. No.: US 2008/0064725 A1
(43) Pub. Date: Mar. 13, 2008

(54) INTRATHECAL ADMINISTRATION OF TRIPTAN COMPOSITIONS TO TREAT NON-MIGRAINE PAIN

(76) Inventors: Allan Basbaum, San Francisco, CA (US); Tetsuro Nikai, San Francisco, CA (US); Andrew Ahn, El Cerrito, CA (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(21) Appl. No.: 11/844,213

(22) Filed: Aug. 23, 2007

Related U.S. Application Data
(60) Provisional application No. 60/823,602, filed on Aug. 25, 2006.

(51) Int. Cl. A61K 31/42; A61K 31/40; A61K 31/405; A61P 25/00; A61K 31/445; A61K 31/41

(52) U.S. Cl. 514/323; 514/376; 514/383; 514/411; 514/414; 514/415

(57) ABSTRACT

Intrathecal delivery of a pharmaceutically acceptable formulation for intrathecal administration of any drug selectively binding to this receptor to provide pain can be used in any situation in which intrathecal (“IT”) drugs are presently used for pain management. In the preferred embodiment, the drug is a triptan. In another embodiment, a combination of drugs with triptans can be used instead of just the triptan. Exemplary conditions to be treated include cancer pain, chronic back pain, post-herpetic neuralgia, and complex regional pain syndrome types I or II, as well as post-traumatic pain, diabetic vasculopathy, inflammatory radiculopathy, inflammatory plexopathies such as brachial plexopathy (Parsonage Turner syndrome), or lumbar plexopathy, HIV neuropathy, chemotherapy-induced neuropathy (such as vincristine toxicity), erythromelalgia, and inherited painful disorders such as metachromatic leukodystrophy, Friedreich's ataxia, and Fabry's disease. The triptans can also be used in acute pain management, such as in labor management or spinal blockade for surgery, where a spinal formulation of sumatriptan could be combined with traditional opiates for synergistic or additive effects.

### PRIORITY

This application claims priority under 35 U.S.C. 119 to U.S. Ser. No. 60/823,602 filed Aug. 25, 2006.

### GOVERNMENT RIGHTS

The United States government may have certain rights in this invention by virtue of grants from the National Institute of Neurological Disorders and Strokes, NS47113 to A. H. Ahn and NS14627 and NS 21445 to A. Basbaum, and NIH-NINDS grant NS 48499.

### FIELD OF THE INVENTION

The present invention is generally in the field of triptan formulations for the treatment of non-migraine pain and methods of use thereof.

### BACKGROUND OF THE INVENTION

Acute pain and chronic pain differ in their etiology, pathophysiology, diagnosis and treatment. Acute pain is self-limiting and serves a protective biological function by acting as a warning of on-going tissue damage. It is a symptom of a disease process experienced in or around the injured or diseased tissue. Associated psychological symptoms are minimal and are usually limited to mild anxiety. Acute pain is nociceptive in nature, and occurs secondary to chemical, mechanical and thermal stimulation of A-delta and C-polymodal pain receptors.

Chronic pain, serves no protective biological function. Rather than being the symptom of a disease process, chronic pain is itself a disease process. Chronic pain is unrelenting, not self-limiting and can persist for years and even decades after the initial injury. Chronic pain can be refractory to multiple treatment modalities. If chronic pain is inadequately treated, associated symptoms can include chronic anxiety, fear, depression, sleeplessness and impairment of social interaction. Chronic, non-malignant pain is predominately neuropathic in nature and involves damage either to the peripheral or central nervous systems.

Nociceptive and neuropathic pain are caused by different neurophysiological processes, and therefore tend to respond to different treatment modalities. Nociceptive pain is mediated by receptors on A-delta and C-fibers which are located in skin, bone, connective tissue, muscle and viscera. These receptors serve a biologically useful role at localizing noxious chemical, thermal and mechanical stimuli. Nociceptive pain can be somatic or visceral in nature. Somatic pain tends to be well localized, constant pain that is described as sharp, aching, throbbing, or gnawing. Visceral pain tends to be vague in distribution, paroxysmal in nature and is usually described as deep, aching, squeezing and colicky in nature. Examples of nociceptive pain include: post-operative pain, pain associated with trauma, and the chronic pain of arthritis. Nociceptive pain usually responds to opioids and non-steroidal anti-inflammatories (NSAIDS).

Neuropathic pain, in contrast to nociceptive pain, is described as “burning,” “electric”, “tingling”, and “shooting” in nature. It can be continuous or paroxysmal in presentation. Whereas nociceptive pain is caused by the stimulation of peripheral of A-delta and C-polymodal pain receptors, by algogenic substances (eg. histamine bradykinin, substance P, etc.) neuropathic pain is produced by damage to, or pathological changes in, the peripheral or central nervous systems. Examples of pathological changes include prolonged peripheral or central neuronal sensitization, central sensitization related damage to nervous system inhibitory functions, and abnormal interactions between the somatic and sympathetic nervous systems. The hallmarks of neuropathic pain are chronic allodynia and hyperalgesia. Allodynia is defined as pain resulting from a stimulus that ordinarily does not elicit a painful response (eg. light touch). Hyperalgesia is defined as an increased sensitivity to a normally painful stimuli. Primary hyperalgesia, caused by sensitization of C-fibers, occurs immediately within the area of the injury. Secondary hyperalgesia, caused by sensitization of dorsal horn neurons, occurs in the undamaged area surrounding the injury. Examples of neuropathic pain include: monoradiculopathies, trigeminal neuralgia, postherpetic neuralgia, phantom limb pain, complex regional pain syndromes and the various peripheral neuropathies. Neuropathic pain tends to be only partially responsive to opioid therapy.

The mechanisms involved in neuropathic pain are complex and involve both peripheral and central pathophysiologic phenomenon. The underlying dysfunction may involve deafferentation within the peripheral nervous system (eg. neuropathy), deafferentation within the central nervous system (eg. post-thalamic stroke) or an imbalance between the two (eg. phantom limb pain).

Following a peripheral nerve injury, sensitization occurs which is characterized by spontaneous activity by the neuron, a lowered threshold for activation and increased response to a given stimulus. Should the injured nerve be a nociceptor, then increased nervous discharge will equate to increased pain. Following nerve injury C-fiber nociceptors can develop new adrenergic receptors and sensitivity, which may help to explain the mechanism of sympathetically maintained pain. In addition to sensitization following damage to peripheral nerves, the formation of ectopic neuronal pacemakers can occur at various sites along the length of the nerve. Increased densities of abnormal or dysfunctional sodium channels are thought to be the cause of this ectopic activity. The sodium channels in damaged nerves differ pharmacologically and demonstrate different depolarization characteristics. This may explain the rationale of treatment with lidocaine, mexiletine, phenyloin, carbamazepine, and tricyclic antidepressants, which block sodium channels. These ectopic pacemakers can occur in the proximal stump (eg. neuroma), in the cell bodies of the dorsal root ganglion, and in focal areas of demylenation along the axon. Neuromas are composed of abnormal sprouting axons and have a significant degree of sympathetic innervation. Neuromas have been reported to accumulate sodium channels at their distal ends which can modulate their sensitivity. They can acquire adrenergic sensitivity, as indicated by increased pain following injection of norepinephrine into the neuroma. Neuromas can also acquire sensitivity to catecholamines, prostanoids and cytokines.

Following a peripheral nerve injury, anatomical and neurochemical changes can occur within the central nervous system (CNS) that can persist long after the injury has healed. This “CNS plasticity” may play an important role in the evolution of chronic, neuropathic pain. As is the case in the periphery, sensitization of neurons can occur within the dorsal horn following peripheral tissue damage. This is characterized by an increased spontaneous activity of the dorsal horn neurons, a decreased threshold and an increased responsivity to afferent input, and cell death in the spinal dorsal horn. The connective tissue sheath around peripheral nerves is innervated by the nervi nervorum. Injury, compression, and inflammation of the sheath may cause pain. In the non-injured state, A beta fibers (large myelinated afferents) penetrate the dorsal horn, travel ventrally, and terminate in lamina III and deeper. C fibers (small unmyelinated afferents) penetrate directly and generally terminate no deeper than lamina II. However, after peripheral nerve injury there is a prominent sprouting of large afferents dorsally from lamina III into laminae I and II. After peripheral nerve injury, these large afferents gain access to spinal regions involved in transmitting high intensity, noxious signals, instead of merely encoding low threshold information. Significant alterations have been shown in the dorsal horn ipsilateral to the injury. The mechanisms are likely related to the barrage of afferent impulses or the factors transported from the lesion site.

Early recognition and aggressive management of neuropathic pain is critical to successful outcome. Oftentimes, multiple treatment modalities are provided by an interdisciplinary management team. Numerous treatment modalities are available and include systemic medication, physical modalities (eg. physical rehabilitation), psychological modalities (eg. behavior modification, relaxation training), invasive procedures (eg. trigger-point injections, epidural steroids, sympathetic blocks), spinal cord stimulators, intrathecal morphine pump systems and various surgical techniques (eg. dorsal root entry zone lesions, cordotomy and sympathectomy). It should be noted that caution is warranted regarding the use of neuroablative techniques. Such approaches may produce deaffrentation and exacerbate the underlying neuropathic mechanisms.

Most neuropathic pain responds poorly to NSAIDS and opioid analgesics. The tricyclic antidepressants (TCA's), the anticonvulsants and the systemic local anesthetics are predominantly the mainstay of treatment. Other pharmacological agents that have proven efficacious include the corticosteroids, topical therapy with substance P depletors, autonomic drugs and NMDA receptor antagonists. The TCA's have been successfully used for the treatment of neuropathic pain for some 25 years. The mechanism of action for the alleviation of neuropathic pain is thought to be due to the inhibition of reuptake of serotonin and norepinephrine within the dorsal horn, however, other possible mechanisms of action include alpha-adrenergic blockade, sodium channel effects and NMDA receptor antagonism.

The selective serotonin reuptake inhibitors (SSRI's) have not proven to be as effective against neuropathic pain as anticipated. Fluoxetine (Prozac) only appears to relieve pain in patients with co-morbid depression. Paroxetine (Paxil™) has found some utility in the treatment of chronic, daily headaches. In general, the SSRI's are partially effective in the treatment of diabetic neuropathy, but not to the extent of the TCA's. Venlafaxine (Effexor™) may have some analgesic effects since, like the TCA's, it inhibits the reuptake of both serotonin and norepinephrine. Its side effect profile is similar to the other SSRI's and can include agitation, insomnia, or somnolence, gastrointestinal distress and inhibition of sexual functioning. Anticholinergic side effects are less bothersome than with the TCA's. The anti-convulsant medications can be effective treatment for neuropathic pain that is described as burning and lancinating in nature. Commonly used medications in this category include phenylytoin, carbamazepine, valproic acid, clonazepam, and gabapentin.

The systemic local anesthetics which are commercially available include lidocaine, tocainide, and mexiletine. The assumed mechanism of action to effect analgesia is the acute blocking of sodium channels. Phenyloin, carbamazepine and tricyclic antidepressants also act as sodium channel blockers. Following the use of the TCA's and anticonvulsants, local anesthetics tend to be third line drugs. Autonomic drugs which may be beneficial in the treatment of neuropathic pain include the alpha-2 agonists (eg. Clonidine) and alpha-1 antagonists (eg. prazosin, terazosin). Dexmedetomidine has affinity to all three alpha 2 adrenergic subtypes. Several other pharmacological treatments which have proven beneficial in the treatment of neuropathic pain include the corticosteroids, and capsaicin cream. Corticosteroids are believed to provide long-term pain relief because of their ability to inhibit the production of phospholipase-A-2 and through membrane stabilizing effects, hence their utility for epidural steroid injections.

If a chronic neuropathic pain condition is already well established, treatment is more difficult. Two agents are currently available. Ketamine is an injectable anesthetic that non-competitively antagonizes NMDA receptors. Although it has proven beneficial in the treatment of neuropathic pain, side effects tend to be unacceptable. NMDA receptor antagonists are known to induce psychomimetic reactions in adult humans and induce behavioral disturbances such as learning and memory impairments, sensorimotor disturbances, stereotypical behavior and hyperactivity and pathomorphological changes in neurons of the posterior cingulate/retrosplenial (PC/RS) cortex of the adult rat. Activation of NMDA receptors leads to calcium entry into the cell and initiates a series of central sensitization. This sensitization may be blocked not only with NMDA receptor antagonists, but also with calcium channel blockers that prevent Ca2+ entry into cells. Clinical experience with the use of opioids for chronic non-malignant pain which is neuropathic in character suggests that there may be a subpopulation of chronic pain patients who may clearly benefit from maintenance with opioid analgesics. Agents that may soon be available for the treatment of neuropathic pain include: 1) butyl-para-aminobensoate (Butamben®), an ester local anesthetic, 2) bupivacaine microspheres, and 3) SNX-III, a selective calcium channel blocker. Nicotinic acetylcholine receptor agonists such as ABT-594, which may also prove efficacious, are in preliminary research stages.

Migraine is more than just pain. Although migraine is usually characterized by headache, the head pain does not uniquely identify migraine. The International Classification of Headache Disorders (Silberstein et al., 2005) defines migraine as a recurrent headache disorder that is accompanied by neurological symptoms, the most common being (a) nausea and vomiting (b) an unpleasant sensitivity to light or sound (c) the presence of other sensory changes such as numbness, tingling, or dizziness, and (d) changes in thinking, wakefulness, or slurred speech. Other variants of migraine include those accompanied by motor weakness, called hemiplegic migraine. The diagnosis of acephalalgic migraine arises from recurrent episodes of these neurological symptoms, but in the absence of headache. The presence of such diverse neurological symptoms that accompany migraine, referred to multiple distinct functional areas of the brain, indicate that migraine is not just a pain disorder, but rather is a global disorder of brain function in which pain is a major feature.

Consistent with this view, the neurobiological features of migraine are not identical to those associated with pain not associated with migraine. Independent studies of brain metabolism, using various imaging techniques, have shown migraine-associated areas of metabolic activity in the brainstem (Weiller et al., Nat Med 1:658-660 (1995)), the hypothalamus (Bahra et al., Lancet 357:1016-1017 (2001)) and the cerebral cortex (Woods et al., N Engl J Med 331:1689-1692 (1994)). These areas are activated in a manner that is not identical to that observed in non-migrainous pain conditions (May et al., Pain 74:61-66 (1998)).

There is a need for additional means for pain management, especially chronic refractory pain and some types of neuropathic pain. There is also a need for an alternative to opioids.

It is therefore an object of the present invention to provide formulations and methods of administration for acute pain.

It is a further object of the present invention to provide formulations and methods of administration which alone or in combination with pain medications such as the opioids, may be useful for treatment of neuropathic pain.

### SUMMARY OF THE INVENTION

Based in part on the discovery of a pain triggered exocytosis and delivery of peptidergic dense core vesicle (“DSV”)-bound 5-HT_{1D} receptor to the plasma membrane, intrathecal delivery of a pharmaceutically acceptable formulation for intrathecal administration of any drug selectively binding to this receptor to provide pain can be used in any situation in which intrathecal (“IT”) drugs are presently used for pain management. In the preferred embodiment, the drug is a triptan. The optimal formulation for intrathecal delivery is a version of Elliot's B artificial CSF, which has been used as a diluent for other intrathecal drugs, such as methotrexate for the treatment of CNS leukemia. In another embodiment, combination of drugs with triptans can be used instead of just the triptan. For example, for chronic refractory pain, IT triptans can be used alone or in combination with traditional IT drugs, such as morphine, clonidine, fentanyl and baclofen.

Sumatriptan and the other triptan drugs target the serotonin receptor subtypes 1B, 1D, and 1F (5-HT_{1B/D/F}), and are prescribed widely in the treatment of migraine. An anti-migraine action of triptans has been postulated at multiple targets, within the brain and at both the central and peripheral terminals of trigeminal “pain-sensory” fibers. However, as triptan receptors are also located on “pain-sensory” afferents throughout the body, it is surprising that triptans only reduce migraine pain in humans, and experimental cranial pain in animals. The examples demonstrate that sumatriptan can reduce non-cranial, somatic pain. Since sumatriptan must cross the blood brain barrier to reach somatic afferent terminals in the spinal cord, systemic delivery was compared to direct spinal (intrathecal) sumatriptan. In tests of acute pain, sumatriptan was without effect, regardless of route. However, in behavioral models of persistent inflammatory pain, a profound analgesic action of intrathecal, but not systemic, sumatriptan was observed. By contrast, sumatriptan was completely ineffective in an experimental model of neuropathic pain, a condition that downregulates 5-HT_{1D} receptors in the spinal cord. The pronounced activity of intrathecal sumatriptan against inflammatory pain demonstrates that there is a wider spectrum of therapeutic indications for triptans beyond headache.

Exemplary conditions to be treated include cancer pain, chronic back pain, post-herpetic neuralgia, and complex regional pain syndrome types I or II, as well as post-traumatic pain, diabetic vasculopathy, inflammatory radiculopathy, inflammatory plexopathies such as brachial plexopathy (Parsonage Turner syndrome), or lumbar plexopathy, HIV neuropathy, chemotherapy-induced neuropathy (such as vincristine toxicity), erythromelalgia, and inherited painful disorders such as metachromatic leukodystrophy, Friedreich's ataxia, and Fabry's disease. Many of the these would be considered neuropathic pains. The triptans can also be used in acute pain management, such as in labor management or spinal blockade for surgery, where a spinal formulation of sumatriptan could be combined with traditional opiates for synergistic or additive effects.

### BRIEF DESCRIPTION OF THE DRAWINGS

FIG. 1 is a diagram of the mechanism of action showing inhibition of transmitter release from the central terminals of primary afferent nociceptors, after nociceptive activity has externalized the 5-HT1D receptor to the plasma membrane, from an intracellular pool associated with DCV's.

FIG. 2 is a graph that demonstrates that Sumatriptan subcutaneous (“SC”)/IT has no effect on locomotor activity

FIGS. 3A and 3B are graphs that show that Sumatriptan is without effect in tests of acute pain.

FIGS. 4A, 4B, 4C and 4D are graphs that show that IT sumatriptan reduces carrageenan-induced hypersensitivity.

FIGS. 5A and 5B are graphs that show that Sumatriptan IT, but not SC, is anti-allodynic in both mechanical and thermal tests after carrageenan (and analgesic in Hargreave's).

FIGS. 6A and 6B are graphs that show that IT sumatriptan completely (and dose-dependently) reverses the allodynia produced by carrageenan

FIG. 7 is a graph that shows that IT sumatriptan inhibits formalin-induced pain behaviors and that Phase 2 inhibition is complete.

FIG. 8A shows that IT, not SC, sumatriptan reduces Phase I pain behavior in the formalin test. FIG. 8B shows that Sumatriptan inhibits Phase 2 pain behavior in the formalin test.

FIGS. 9A, 9B and 9C are graphs of the results of tests of nociception by (FIG. 9A) hot-plate test, and (FIG. 9B) radiant heat to the hindpaw (Hargreaves test) (seconds latency), or (FIG. 9C) mechanical pain using calibrated monofilaments (g threshold), demonstrate no significant changes in threshold over the time course of the test after systemic (SC) or intrathecal (IT) administration of sumatriptan. SC doses were at 300 μg/kg (SC300) and 600 μg/kg (SC600), and IT doses were 0.06 μg (IT0.06) and 0.60 μg (IT0.60). A positive control of 10 nmol IT morphine sulfate (MSO₄) produced a robust analgesic response in these tests. FIG. 9D is a graph of % of baseline nociceptive effect, showing lack of a nociceptive effect at the doses administered in these studies.

FIGS. 10A and 10B are graphs of response (seconds) over time showing that intrathecal sumatriptan selectively and profoundly reduces the second phase of formalin-induced pain. The formalin test began one hour after the administration of saline, sumatriptan, or morphine. The time course of hindpaw licking in 5 min bins (FIG. 10A), and the cumulative time spent licking in phase 1 (0-10 min) and phase 2 (11-60 min) (FIG. 10B) show that both IT saline- and SC sumatriptan-injected animals displayed stereotypical biphasic behaviors, but that only intrathecal (IT) administration of sumatriptan selectively and dose-dependently reduced the amount of second phase behaviors. SC doses of sumatriptan were 300 μg/kg (SC300) and 600 μg/kg (SC600). IT doses of sumatriptan were 0.006 μg (IT0.006), 0.06 μg (IT0.06) and 0.60 μg (IT0.60). A positive control of 10 nmol IT morphine sulfate (MSO₄) produced a robust analgesic response in this test.

FIGS. 11A, 11B, 11C and 11D are graphs showing that sumatriptan modulates inflammation-induced hypersensitivity over time in minutes when given intrathecally. The time-course of sumatriptan responses after sensitization by carageenan is shown to thermal (FIG. 11A) and mechanical (FIG. 11B) stimulation. The pre-test baseline is shown at left (pre) Thermal (FIG. 11A) and mechanical (Figure B) thresholds are greatly reduced at 24 hours after injection of carrageenan to the left hindpaw (carra). Responses are shown over a time course (from 30 to 240 min) for a range of doses after the administration of or IT sumatriptan. Responses of the contralateral hindpaw (contra) remained unchanged throughout the procedure. Reduction of thermal (FIG. 11C) and mechanical (FIG. 11D) hyperalgesia by IT sumatriptan is dose-dependent, shown at 30-90 min after administration of drug (doses are same as in FIG. 10). Values are given as percent of the maximal possible effect (% MPE).

FIG. 12. Responsiveness to sumatriptan correlates with changes in 5-HT_{1D} receptor expression at the central terminals of nociceptive afferents, shown as threshold (g) for spared nerve injury of the sciatic nerve, a mechanical hyperalgesia ipsilateral to the injury that is stable and fully developed at 7 days post-nerve transection (SNI 7d). Neither IT sumatriptan 0.6 μg (IT suma) nor IT saline (IT saline) reduced SNI-induced hypersensitivity, 30-120 min after administration of drug, whereas IT morphine (IT morphine) produced a significant analgesia. Nociceptive thresholds of the unaffected contralateral leg (contra) are unaffected by the treatment of saline or sumatriptan.

### DETAILED DESCRIPTION OF THE INVENTION

I. Compositions

As used herein, “alkyl” refers to alkyl, alkenyl, and alkynyl groups. Examples of alkyl groups include methyl, ethyl, propyl, isopropyl, butyl, iso-butyl, sec-butyl, pentyl, 3-pentyl, hexyl, heptyl, octyl and the like. Examples of alkenyl groups include ethenyl, propenyl, butenyl, pentenyl, hexenyl, and the like. Examples of alkynyl groups include ethynyl, propynyl, butynyl, pentynyl, hexynyl, and the like. The number of carbons in the alkyl group is from 1 to 20, preferably from 1-10, and more preferably from 1-8.

As used herein, the term “cycloalkyl” can be bicycloalkyl (norbornyl, 2.2.2-bicyclooctyl, etc.) and tricycloalkyl (adamantyl, etc.), optionally including 1-2 N, O or S atoms. Cycloalkyl also encompasses (cycloalkyl)alkyl. The number of carbon atoms in the cycloalkyl group is from 3 to 10, preferably from 3-8, and more preferably from 3-6.

As used herein, the term “aryl” includes phenyl, indenyl, indanyl, naphthyl, and the like. In addition, aryl includes ortho-fused bicyclic carbocyclic radicals having about nine to ten ring atoms in which at least one ring is aromatic. The term “aryl” can include radicals of an ortho-fused bicyclic heterocycle of about eight to ten ring atoms derived therefrom, particularly a benz-derivative or one derived by fusing a propylene, trimethylene, or tetramethylene diradical thereto.

As used herein, the term “heteroaryl” can be a monocyclic aromatic ring containing five or six ring atoms consisting of carbon and 1, 2, 3, or 4 heteroatoms each selected from the group consisting of non-peroxide oxygen, sulfur, and N(Y) where Y is absent or is H, O, alkyl, phenyl or benzyl. Non-limiting examples of heteroaryl groups include furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide), quinolyl (or its N-oxide) and the like. The term “heteroaryl” can include radicals of an ortho-fused bicyclic heterocycle of about eight to ten ring atoms derived therefrom, particularly a benz-derivative or one derived by fusing a propylene, trimethylene, or tetramethylene diradical thereto. Examples of heteroaryl can be furyl, imidazolyl, triazolyl, triazinyl, oxazoyl, isoxazoyl, thiazolyl, isothiazoyl, pyraxolyl, pyrrolyl, pyrazinyl, tetrazolyl, pyridyl (or its N-oxide), thientyl, pyrimidinyl (or its N-oxide), indolyl, isoquinolyl (or its N-oxide), quinolyl (or its N-oxide), and the like.

As used herein, an “analog” of a chemical compound is a compound that, by way of example, resembles another in structure but is not necessarily an isomer (e.g., 5-fluorouracil is an analog of thymine).

As used herein, a “derivative” of a compound refers to a chemical compound that may be produced from another compound of similar structure in one or more steps. Derivatives generally involve the addition, deletion, and/or modification of one or more functional groups on the parent compound.

As used herein, the term “stereoisomers” refers to corn pounds made up of the same atoms bonded by the same bonds but having different spatial structures which are not interchangeable. The three-dimensional structures are called configurations. As used herein, the term “enantiomers” refers to two stereoisomers whose molecules are nonsuperimposable mirror images of one another. As used herein, the term “optical isomer” is equivalent to the term “enantiomer”. The terms “racemate”, “racemic mixture” or “racemic modification” refer to a mixture of equal parts of enantiomers. The term “chiral center” refers to a carbon atom to which four different groups are attached, as distinguished from prochiral centers. The term “enantiomeric enrichment” as used herein refers to the increase in the amount of one enantiomer as compared to the other. Enantiomeric enrichment is readily determined by one of ordinary skill in the art using standard techniques and procedures, such as gas or high performance liquid chromatography with a chiral column. Choice of the appropriate chiral column, eluent and conditions necessary to effect separation of the enantiomeric pair is well within the knowledge of one of ordinary skill in the art using standard techniques well known in the art, such as those described by J. Jacques, et al., “Enantiomers, Racemates, and Resolutions”, John Wiley and Sons, Inc., 1981. Examples of resolutions include recrystallization of diastereomeric salts/derivatives and/or preparative chiral chromatography.

A. Triptans

The compositions described herein contain one or more triptans, analogues or derivatives thereof, and/or pharmaceutically acceptable salts thereof. In one embodiment, the triptan has the structure of formula I:

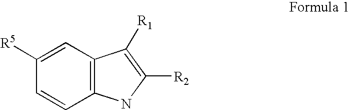

wherein R₁ and R₂ are independently hydrogen; linear, branched, or cyclic alkyl; substituted linear, branched, or cyclic alkyl; linear, branched, or cyclic heteroalkyl; substituted linear, branched, or cyclic heteroalkyl; or wherein R₁ and R₂ together formed a fused ring having 4-10 atoms, wherein the fused ring is optionally substituted at one or more positions; and R₃ is hydrogen; linear, branched, or cyclic alkyl; substituted linear, branched, or cyclic alkyl; linear, branched, or cyclic heteroalkyl; substituted linear, branched, or cyclic heteroalkyl; aryl, substituted aryl,

Examples of suitable triptans having the structure of formula I, include, but are not limited to, rizatriptan, eletriptan, naratriptan, zolmitriptan, frovatriptan, sumatriptan, almotriptan, and combinations thereof. The structures of these triptans are show below.

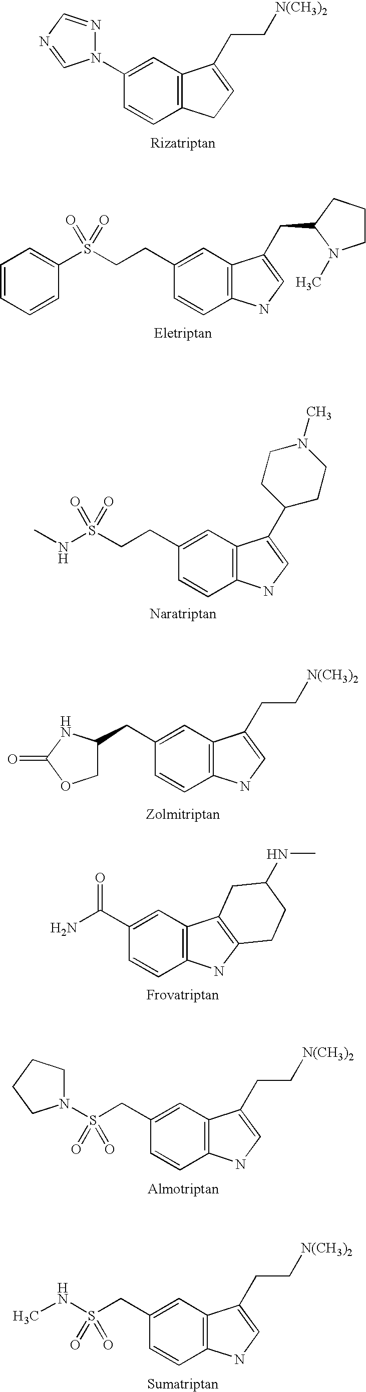

Other suitable triptans include, but are not limited to, PNU-109291, GR 127935, LY344864, and PNU-142633F.

The compounds may be administered as the free base. However, the compounds are typically administered as a pharmaceutically acceptable acid-addition salt. As used herein, “Pharmaceutically acceptable salts” refer to derivatives of the compounds wherein the parent compound is modified by making the acid addition salt thereof. Examples of pharmaceutically acceptable acid-addition salts include, but are not limited to, mineral or organic acid salts of basic residues such as amines. The pharmaceutically acceptable salts include the conventional non-toxic salts or the quaternary ammonium salts of the parent compound formed, for example, from non-toxic inorganic or organic acids. Such conventional non-toxic salts include, but are not limited to, those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, naphthalenesulfonic, methanesulfonic, ethane disulfonic, oxalic, and isethionic salts.

The pharmaceutically acceptable salts of the compounds can be synthesized from the parent compound, which contains a basic moiety, by conventional chemical methods. Generally, such salts can be prepared by reacting the free base forms of these compounds with a stoichiometric amount of the appropriate acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704; and “Handbook of Pharmaceutical Salts: Properties, Selection, and Use,” P. Heinrich Stahl and Camille G. Wermuth, Eds., Wiley-VCH, Weinheim, 2002.

As generally used herein “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

B. Combinations with Other Active Agents

The triptans may be administered adjunctively with other active compounds. For example, for chronic refractory pain, IT triptans can be used alone or in combination with traditional IT drugs, such as an opiate like morphine, clonidine, fentanyl and baclofen; gabapentin/pregabalanin; and calcium channel blockers that can be administered intrathecally including, not limited to, ziconatide, diltiazem, verapamil, SNX-111, and P-conotoxin.

By adjunctive administration is meant simultaneous administration of the compounds, in the same dosage form, simultaneous administration in separate dosage forms, and separate administration of the compounds.

C. Carriers, Additives, and Excipients

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes, but is not limited, to diluents, buffers, salts, and preservatives or stabilizers. Stabilizers are used to inhibit or retard drug decomposition reactions which include, by way of example, oxidative reactions.

The optimal formulation for intrathecal delivery is a version of Elliot's B artificial CSF, which has been used as a diluent for other intrathecal drugs, such as methotrexate for the treatment of CNS leukemia.

II. Disorders to be Treated

The formulations are used to treating pain by administering intrathecally to a patient in need thereof an effective amount of a triptan to treat non-migrainous tissue pain when administered intrathecally, in combination with a pharmaceutically acceptable carrier for intrathecal administration.

In a preferred embodiment, the triptan is rizatriptan, eletriptan, naratriptan, zolmitriptan, frovatriptan, sumatriptan, almotriptan, or a combination thereof. The triptan may be administered in combination with a second agent for pain control, in the same formulation or by injection or oral administration of the second agent. In preferred embodiments, the second agent is an opiate such as morphine, clonidine, fentanyl or baclofen. The triptan may also be administered in combination with a drug such as gabapentin or pregabalin.

The examples demonstrate that the intrathecal triptan is highly effective at treating inflammatory pain. Examples of disorders causing pain that can be treated include cancer pain, chronic back pain, rheumatoid arthritis, osteoarthritis, post-herpetic neuralgia, and complex regional pain syndrome types I or II, post-traumatic or post-operative pain, diabetic vasculopathy, inflammatory radiculopathy, and inflammatory plexopathies such as brachial plexopathy (Parsonage Turner syndrome) or lumbar plexopathy. Although not demonstrated to have significant efficacy in the animal model described in the examples, it is expected that the drug will be used to treat neuropathic pain in humans, for example, resulting from any of the following conditions: HIV neuropathy, chemotherapy-induced neuropathy (such as vincristine toxicity), erythromelalgia, diabetic neuropathy, and inherited painful disorders such as metachromatic leukodystrophy, Friedreich's ataxia, and Fabry's disease.

The intrathecal triptan is useful for acute pain management. It can also be used to treat pain secondary to spinal cord injury and for labor management or spinal blockade for surgery.

As noted above, the preferred method of administration is by intrathecal administration. The effective dosage can be calculated based on the studies described in Example 2 below, by those skilled in the art using routine experimentation.

The present invention will be further understood by reference to the following experiments.

### EXAMPLE 1

### Tissue Injury Regulates Serotonin 1D Receptor Expression

Ahn and Basbaum, J. Neurosci. 26(32):8332-8332 (August 2006) reported that the anti-migraine action of “triptan” drugs involves the activation of serotonin subtype 1D (5-HT1D) receptors expressed on “pain-responsive” trigeminal primary afferents. In the central terminals of these nociceptors, the receptor is concentrated on peptidergic dense core vesicles (DCVs) and is notably absent from the plasma membrane. Based on this arrangement, it was hypothesized that in the resting state the receptor is not available for binding by a triptan, but that noxious stimulation of these afferents could trigger vesicular release of DCVs, thus externalizing the receptor. Studies demonstrated that within 5 minutes of an acute mechanical stimulus to the hindpaw of the rat, there is a significant increase of 5-HT1D-immunoreactivity (IR) in the ipsilateral dorsal horn of the spinal cord. These rapid immunohistochemical changes reflect redistribution of sequestered receptor to the plasma membrane, where it is more readily detected. Divergent changes were also observed in 5-HT1D-IR in inflammatory and nerve-injury models of persistent pain, occurring at least in part through the regulation of 5-HT1D-receptor gene expression. 5-HT1D-IR is unchanged in the spinal cord dorsal horn of mice with a deletion of the gene encoding the neuropeptide substance P. This result differs from that reported for the partial differential-opioid receptor, which is also sorted to DCVs, but is greatly reduced in preprotachykinin mutant mice. The results demonstrate a “pain”-triggered regulation of 5-HT1D-receptor expression underlies the effectiveness of triptans for the treatment of migraine. Moreover, the widespread expression of 5-HT1D receptor in somatic nociceptive afferents suggests that triptans could be administered to treat pain in nontrigeminal regions of the body.

Materials and Methods

Receptor Activation in Models of Pain

Male Sprague Dawley rats weighing 175-250 g were used in accordance with protocols approved by the Institutional Animal care and Use Committee.

Noxious mechanical stimulation. Rats were anesthetized under 1.5-2% isorlurane with 2 L/min flow of oxygen until blink and withdrawal reflexes were suppressed. The mechanical pinch stimulus of the left hindpaw was made by 2 min of pressure across the left hindpaw with a loose hemostat; this stimulus evokes the release of substance P from the nociceptors (McCarson and Goldstein, 1991) and internalization of the neurokinin 1 (NK-1) receptor in dorsal horn neurons (Abbadie et al., 1997). The rats were maintained under inhalation anesthesia, until they received a terminal does of pentobarbital (100 mg/kg) and were perfused as described below for immunohistochemistry. Six to nine animals were used for cash time point.

Complete Freund's adjuvant-induced inflammation. To induce tissue inflammation, we injected 75 μl of a 50% emulsion of complete Freund's adjuvant (CFA) (Sigma, Saint Louis, Mo.), mixed in saline, intradermally into the left hindpaw using a 30-gauge needle while animals were anesthetized under 2% isoflurane with L/min flow of O₂. After recovery from anesthesia, the animals were returned to their home cage. From 1-7 d later, the animals were anesthetized and perfused for immunohistochemistry or RNA analysis. Three or six animals were used for each time point.

Sciatic nerve section. In another group of rats, we transected the sciatic nerve under the same inhalation anesthesia protocol. After 21 d., three (n+3) animals were anesthetized and perfused for immunohistochemistry and for RNA analysis.

Immunohistochemistry

Tissue preparation. Animals were overdosed with sodium pentobarbital (100 mg/kg) and perfused with heparnized saline followed by fixation with 10% formalin in 0.1 m sodium phosphate buffer, pH 7.4. For immunostaining, 50 μm transverse frozen sections were cut through the lumbar enlargement. In the 5 min postpinch series, 20 alternate sections were processed from the L4 through the L6 segments. For the longer time points, only sections through the L5 to L6 segments were processed, in which changes in activation of 5-HT_{1D} receptor predominated. The referred region of lumbar spinal cord from CFA treated (L5-L6) and sciatic nerve out (L4-L5) animals were examined in a similar manner.

HRP-DAB immunostaining procedure. Before exposure to antibody, free-floating sections were preincubated for 1 h at room temperature. (RT) in PBS with 0.3% Trinton X-100 (PBST) and 10% normal goat serum (NGS). Primary and secondary antisera were diluted in PBST with 2% NGS (2% NGST). Tissue was incubated overnight at room temperature in rabbit anti-5-HT_{1D} antibody (1:100,000). This affinity-purified antibody, which has been characterized and described in detail previously (Potrebic et al., 2003), was raised against an oligopeptide corresponding to a subtype-specific region of 5-HT_{1D}, predicted to be in an intracellular loop of the receptor. Sections were then washed three times in 2% NGST for 10 min each and incubated for 1 h at room temperature with biotinylated goat anti-rabbit antibody (Vector Laboratories, Burlingame, Calif.) in 2% NGST, and washed three times in PBST for 10 min each at RT. To localize the secondary antibody, an avidin-biotin HRP protocol was used with an ABC kit (Vector Laboratories), glucose oxidase, and nickel-enhanced 3,-3′ diaminobenzidine (DAB; Sigma) as chromogen. Sections were then mounted on gelatin-coated glass sides and coverslipped under DPX mounting media (EM Sciences, Fort Washington, Pa.).

Fluorescence immunohistochemistry. Tissue was fixed and cryoprotected as above. Spinal cord and DRG tissues were cut at 14 μm and stained essentially as described previously (Potrebic et al., 2003). The antibodies were used in the following dilutions: rabbit anti-5-HT_{1D} at 1:40,000; guinea pig anti-substance Pat 1:6000; mouse monoclonal antineurofilament of 200 kDa NF200, clone N52/RT97; Sigma) at 1:1200; mouse monoclonal anti-CGRP (#4901: kindly provided by Dr. Caria Sternin, University of California, Los Angeles, Calif.) at 1:1250 (Wong et al., 1993); rabbit anti-6 opioid receptor (DOR; #10271; Abscam, Cambridge, Mass.) at 1:2000.

Light Microscopy and Image Analysis

Sections with photographed Nikon (Tokyo, Japan) Eclipse microscope with an attached Spot or Zeiss (Oberkochen, Germany) digital camera. 8-10 sections from each animal were analyzed, from files in which the side of the lesion was blinded to the observer. The staining of 5-HT_{1D}-IR with National Institutes of Health image analysis software (Image J for Macintosh OSX) was quantified, by outlining the region of interest in the medial dorsal horn symmetrically on either side of the spinal cord, and then obtaining the mean optical density of the immunohistochemical reaction product. No threshold cutoff was made. The mean optical density of the nearby deep dorsal horn on each side was measured to correct for local illumination effects and variation in background staining, where there was no reaction product, was subtracted. The staining density is expressed as a ratio of the ipsilateral over the contralateral side. All ratios are represented as the mean of the average ratios determined for each animal ±SEM. A Student's t test between the ipsilateral and contralateral sides was applied as a test of significance (P<0.05).

RNA Isolation and Real-Time PCR Analysis

To prepare RNA for analysis, individual lumbar L5 DRG's were collected after intracardiac perfusion with 100 cc saline, followed by 100 cc RNAlater (Ambion, Austin, Tex.). The DRG's were incubated at 4° C. overnight in RNAlater and then stored at −20° C. For RNA isolation, the RNAlater was aspirated and the DRGs homogenized in Trizolt (Invitrogen, Carlsbad, Calif.) with individual disposable microcentrifuge postles, and the isolation procedure was performed per the manufacturer's recommendations. To remove contaminating genomic DNA, the RNA was purified in RNeasy mini-columns (Qiagen, Valencia, Calif.) per the manufacturer's recommendations. The RNA was quantified by Ribogren fluorescence against a standard curve, and the absence of genomic DNA was confirmed by Picogreen fluorescence (Invitrogen). The threshold for detection was less than 10 pg/μl. Three rats were used for each time point, and approximately 1.0 μg of total RNA was isolated from each ganglion. The RNA sample from each ipsilateral ganglion was paired to the contralateral ganglion for comparison of relative expression.

For cDNA synthesis, a parallel set of reactions using 1.0 μg of RNA each was reverse-transcribed with oligo-dT primers and Superscript II reverse transcriptase (Invitrogen), corresponding to a 1.0 μg equivalent of cDNA. After the appropriate dilution curves, 20 ng of cDNA was amplified in a TaqMan® assay using Amplitaq-Gold® reagents (Applied Biosystems, Foster City, Calif.) in an Applied Biosystems Real-Time PCR system. The reactions were all performed in triplicate, and the mouse glyceraldehydes-3phosphate dehydrogenase (GAPDH) primer set (Applied Biosystems) was used as an endolgenous control to normalize the cDNA templates. The 5-HT_{1D} mRNA was detected with flanking primers 5′-CCCGGAGTCGAATCCTGAA-3′ (SEQ ID NO: 1), 5′TGATAAGCTGTGCTGTGGTGAA-3′ (SEQ ID NO: 2), and probe 5′6-FAM-CTATCTTGGTCATGCCCATCAGC-BHQ-3′ (SEQ ID NO: 3) labeled with 6-FAM (6 carboxy fluorescein-aminohexylamidite) and BHQ (black hole quencher; Biosearch Technologies, Novato, Calif.).

Dilution curves were performed on cDNA from pooled DRG or trigeminal ganglion samples in a separate series of reactions to show that this primer set amplified with an efficiency of 97%. The threshold values were determined and performed relative quantification calculations using Applied Biosystems software. Ratios of the individual DRGs compared with their contralateral controls are shown as means ±SEM. A Student's t test between the ipsilateral and contralateral sides was applied as a test of significance (p<0.05).

Results

Acute Activation by Mechanical Pinch

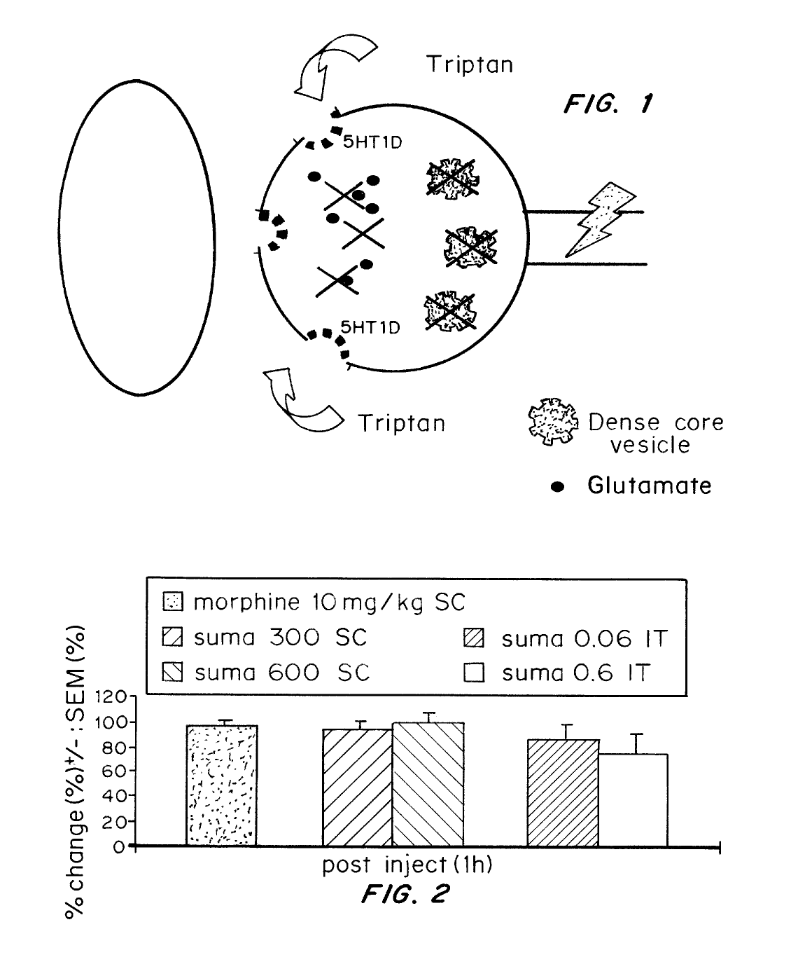

Because noxious mechanical stimulation (pinch) efficiently stimulates substance P release and postsynaptic NK-1 receptor internalization within 5 min, this time point was chosen for acute activation. In animals perfused 5 min after pinch, a significant increase in 5-HT_{1D}-IR in the medial half of the ipsilateral L5 spinal cord dorsal horn, where afferent terminals from the hindpaw are most densely concentrated, was observed (FIG. 1A). To normalize the fixation and immunohistochemical reaction conditions, the staining on the stimulated side was compared to the uncreated contralateral side. In addition to increased density of reaction product, the staining pattern in the ipsilateral dorsal horn was more granular and puncrate ((FIG. 1A). Changes at the L4 segment of the cord were not statistically significant. FIG. 2 summarizes the time course of these changes. When tissue was analyzed 30 min after the pinch stimulus, a significant decrease of 5-HT_{1D}-IR between the two sides was observed.

5-HT_{1D} Receptor in the Setting of Persistent Inflammation

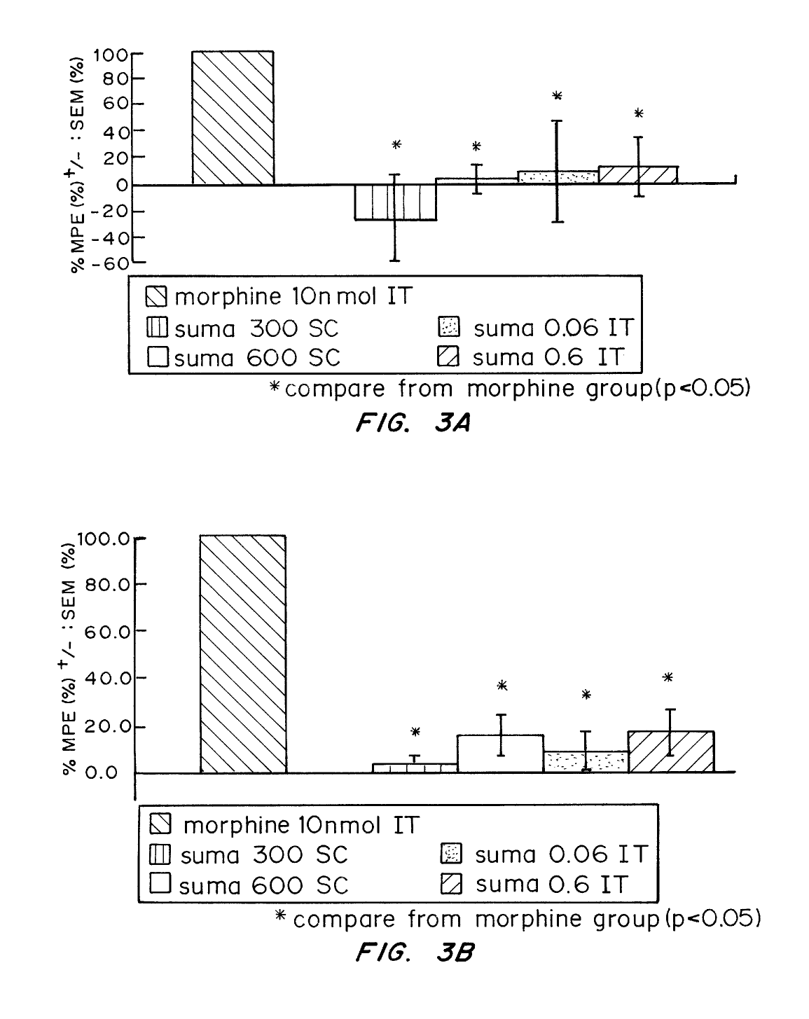

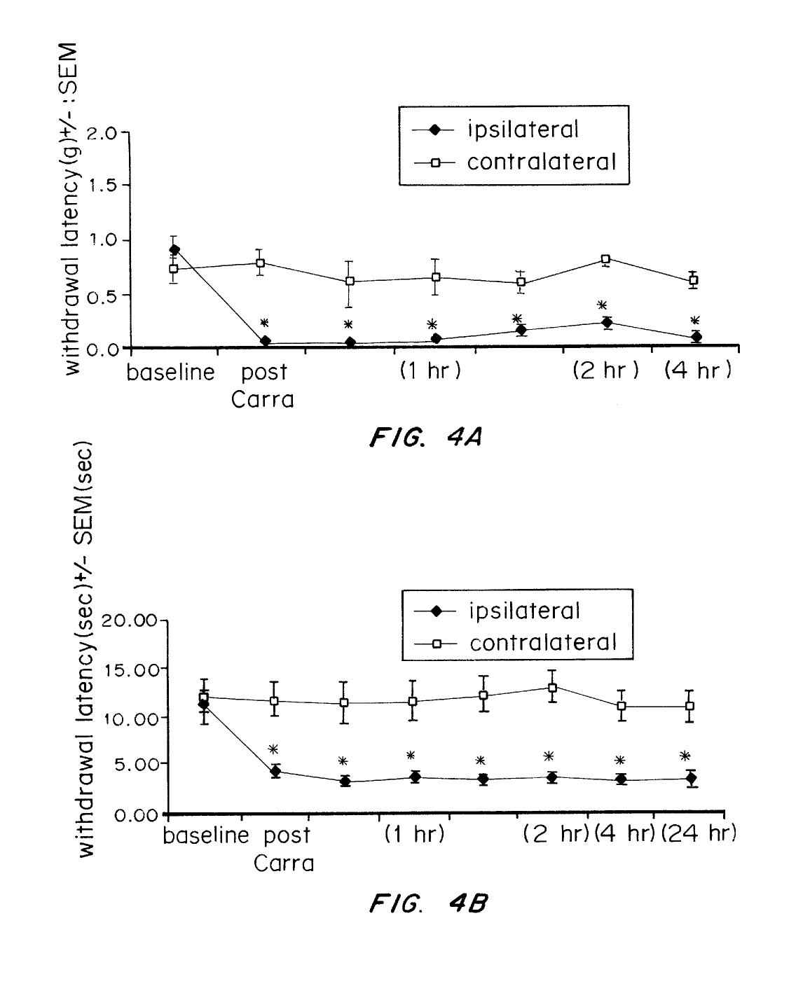

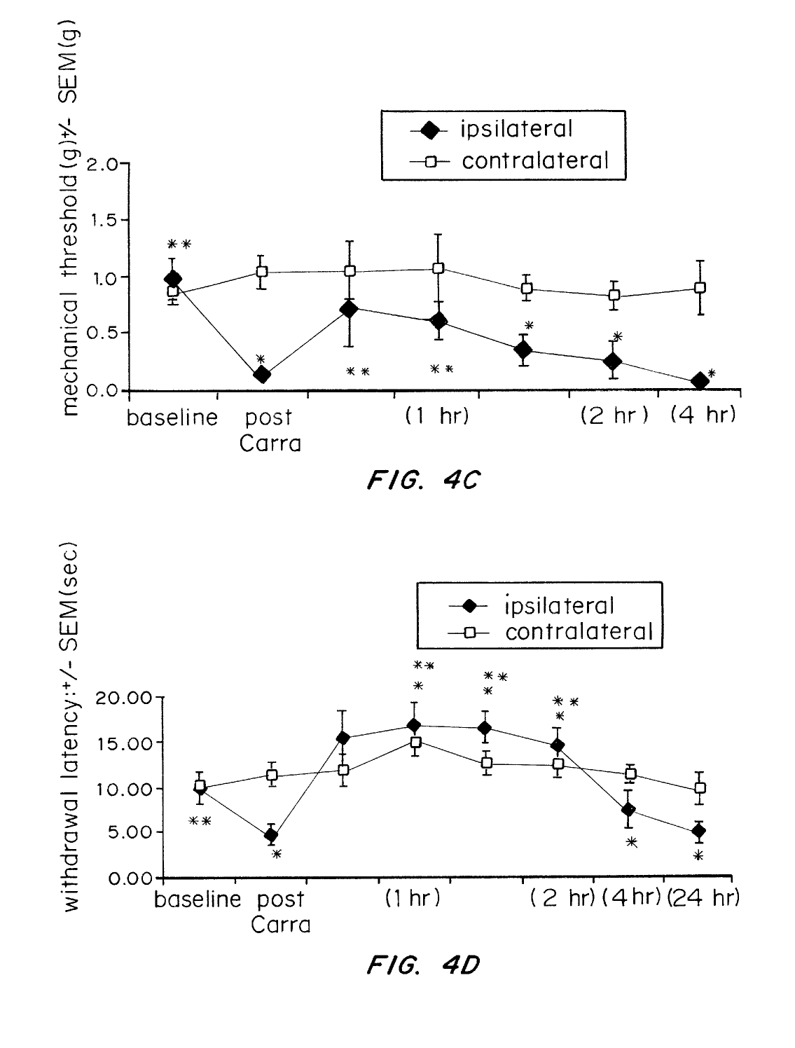

Noxious stimulation with intraplantar CFA involves inflammation and pain-related behaviors that peak at 3 d postinjection, followed by a slow decline in sensitization over the following 10-14 d. A complex progression of 5-HT_{1D}-IR in the 7 d post-injection was observed (FIG. 3), which is quantitatively summarized in FIG. 4. During the first 2 d postinjection, there was a variable degree of expression, both up or down within individual animals that was not significantly different from the contralateral side. On the third postinjection day, 5-HT_{1D}-IR declined significantly compared with the contralateral dorsal horn (FIG. 3A). At 7 d after injection, when paw edema and nociceptive thresholds begin to normalize, a significant increase in 5-HT_{1D}-IR (FIG. 3B) was observed.

The increased 5-HT_{1D}-IR 7 d after CFA could be attributable to either even greater levels of receptor expression in peptidergic afferents, or alternatively, to new synthesis in a separate class of afferents. To address this question, double-label immunohistochemistry was used to determine what proportion of 5-HT_{1D}-IR DRG neurons also expressed the neuropeptide substance P (FIG. 3C). 94% of the ipsilateral 5-HT_{1D}-immunoreactive L5 afferents were also SP immunoreactive, compared with 92% of the contralateral afferents. To address the possibility that 5-HT_{1D}-IR neurons are newly synthesized by myclinated afferents, double-label immunohistochemistry was used for NF200. Only 6% of the 5-HT_{1D}-IR DRG neurons expressed N52 ipsilateral to the stimulus, which was comparable to that on the contralateral side (10%) (FIG. 3D) and comparable with those reported previously in normal untreated animals.

5-HT_{1D}-IR in the Setting of Nerve Injury

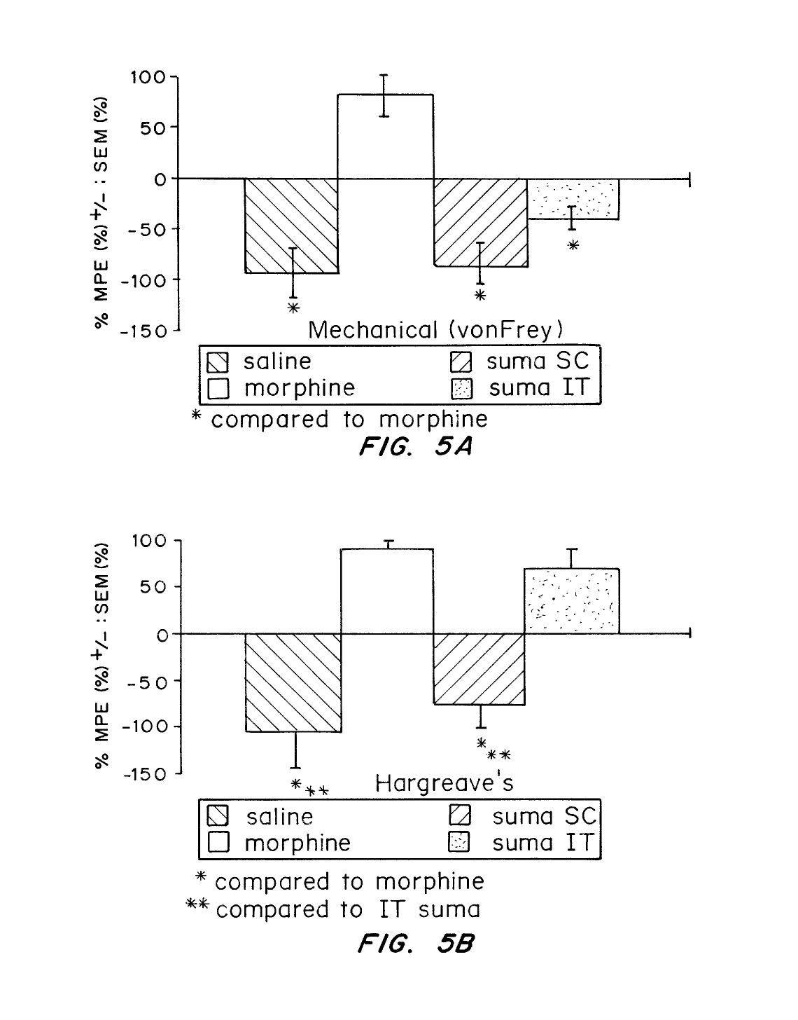

To assess the consequences of nerve injury on 5-HT_{1D} receptor expression, the effects of complete transaction of the sciatic nerve were assessed. This injury produced a dramatic reduction of 5-HT_{1D}-IR in the ipsilateral spinal cord dorsal horn 3 weeks after the surgery (FIG. 5A). Mean optical density of the affected dorsal horn was 10% of the contralateral side. To determine whether there was a concomitant change in the DRG cell bodies that give rise to these afferents, L5 DRG sections were double labeled for 5-HT_{1D} and for a marker of peptidergic afferents. CGRP rather than substance P immunoreactivity (IR) was monitored as the later is almost undetectable in DRG after sciatic nerve transaction. In contrast to the decreased immunoreactivity of the central terminals, persistent 5-HT_{1D}-IR was found in peptidergic afferents of the L5 DRG ipsilateral to the nerve injury (FIG. 5B, C).

5-HT_{1D} Gene Expression in Models of Chronic Pain

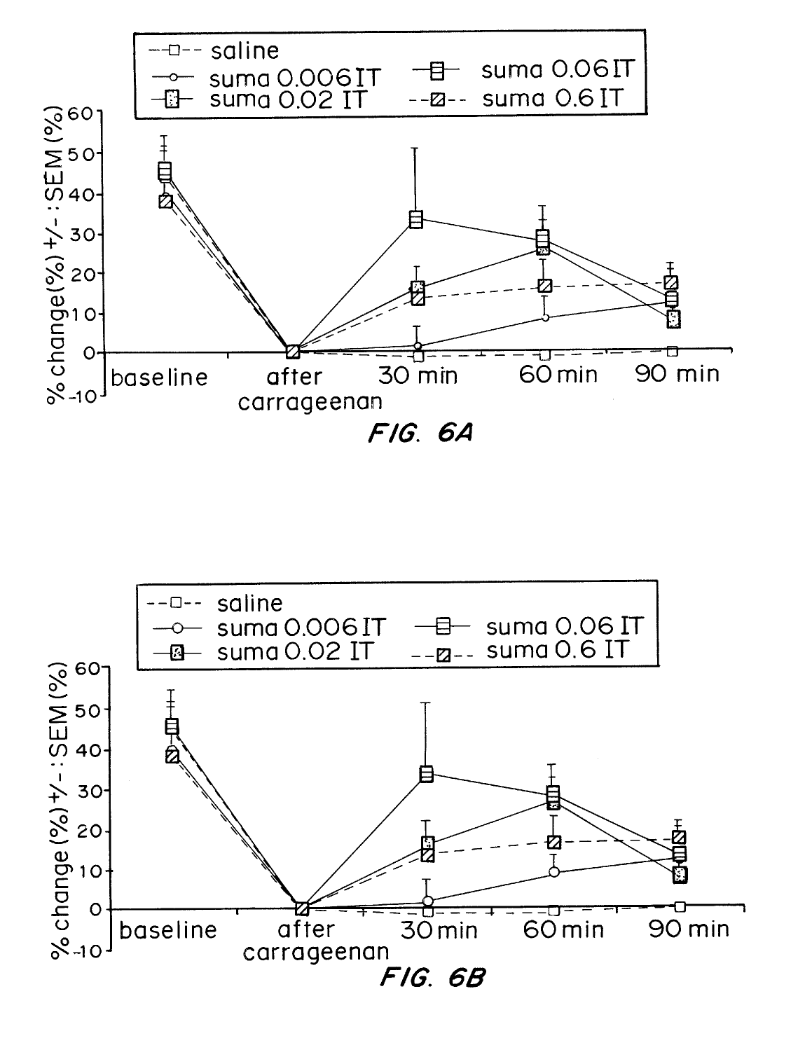

Although the very rapid changes in 5-HT_{1D}-IR produced by acute noxious stimulation likely reflect redistribution of the receptor at the central terminal of the primary afferent nociceptor, the changes observed in the setting of persistent injury could also result from changes in 5-HT_{1D} gene expression in DRG neurons. To address this possibility, a quantitative real-time PCR assay to determine 5-HT_{1D}-mRNA levels in RNA isolated from individual L5 DRs was developed (FIG. 6A). Normalized to the endogenous CAPDH expression levels, the relative abundance of 5-HT_{1D}-mRNA in DROs ipsilateral to the stimulus compared with the unstimulated contralateral L5 DRG was determined. In the CFA model of chronic inflammatory pain, the ipsilateral 5-HT_{1D}-mRNA levels were also significantly elevated compared with the contralateral side.

Targeting 5-HT_{1D}-IR to DCVs

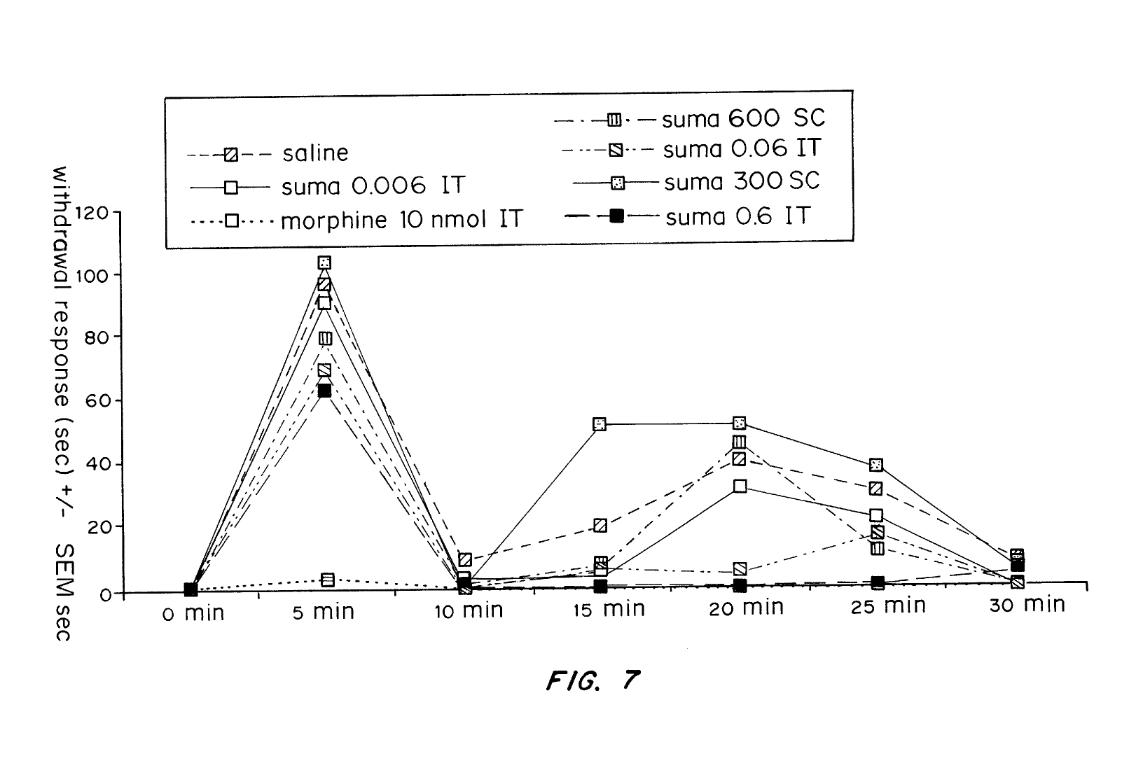

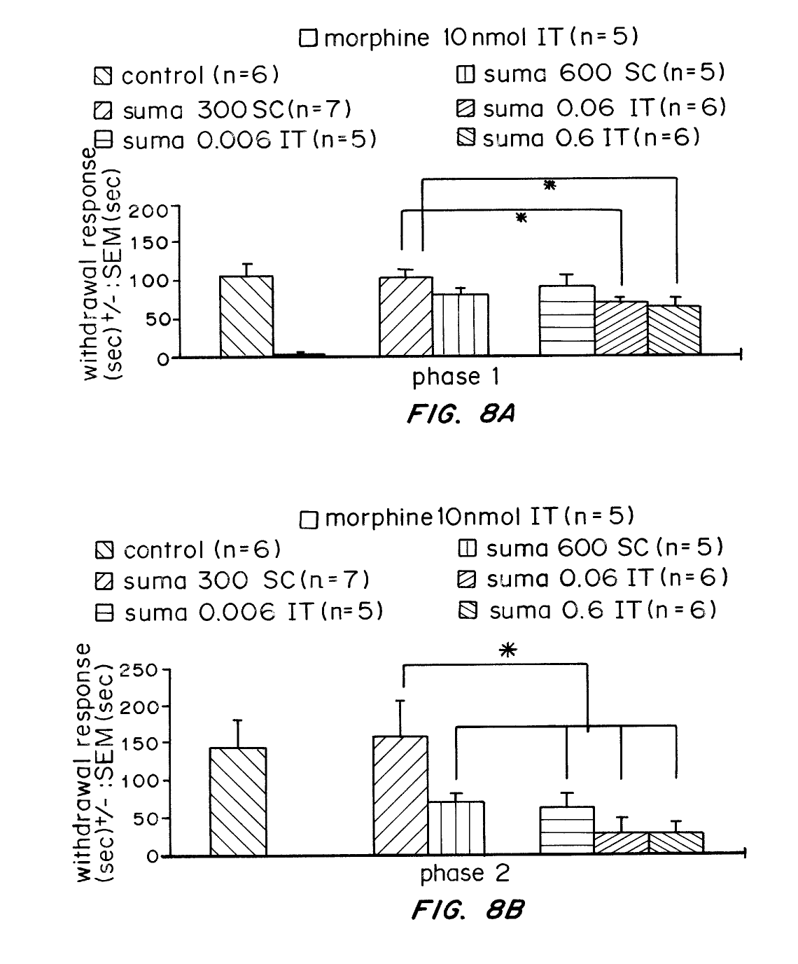

The localization of the 5-HT_{1D} receptor within peptidergic nociceptive terminals is strikingly similar to that of the DOR, raising the possibility that these two receptors share a common sorting mechanism to DCVs. To determine whether the 5-HT_{1D} receptor is regulated in a manner comparable with that of the DOR, DOR, and 5-HT_{1D} receptor-IR was determined in the spinal cords from a line of PPT-A mutant mice. A significant decrease of DOR-IR compared with wild-type littermates (FIG. 1D, F) was found, but in contrast to the DOR, the pattern and magnitude of 5-HT_{1D}-IR did not differ in the PPT-A mutant and their wild-type littermates (FIG. 7B, C). A noxious mechanical stimulus produces rapid and complex change in the magnitude of 5-HT_{1D}-IR in the spinal cord dorsal horn. In a tissue-injury model of persistent pain, such complex changes in 5-HT_{1D}-IR in afferent terminals occurred in parallel with changes in 5-HT_{1D} receptor gene expression. However, despite there being significantly elevated 5-HT_{1D} receptor gene expression in the DRG after sciatic nerve injury, a marked reduction of 5-HT_{1D}-IR in afferent terminals was found, providing evidence for a dissociation between somatic and terminal expression after nerve injury.

Acute Activation-Induced Changes in 5-HT_{1D}-IR

The significant and rapid rise in 5-HT_{1D}-IR, within 5 min of noxious mechanical stimulation, most likely reflects synaptic events within the primary afferent terminal rather than the transport of new receptor to the terminal. Given the close association between substance P and 5-HT_{1D} receptor in primary afferent terminals, one possible explanation for the activity-triggered increase in 5-HT_{1D}-IR is that nociceptor activation redistributes 5-HT_{1D} receptor-bound vesicles to the plasma membrane where the receptor is presumably more efficiently recognized by the antibody.

The biphasic changes in 5-HT_{1D}-IR observed over time, from minutes to hours after stimulation, could be caused by a variety of synaptic and cellular processes Internalization and degradation of the receptor within lysosomes could account for the relative loss of 5-HT_{1D}-IR at 30 min. Alternatively, association with β-arrestin-mediated clathrin-coated pits during vesicular recycling may interfere with the detection to the intracellular normalization of 5-HT_{1D}-IR at 90 min. is compatible with the repletion of 5-HT_{1D} receptor containing DCVs after axoplasmic transport from cell bodies in the DRG.

This model of 5-HT_{1D} receptor delivery to the plasma membrane is directly analogous to the activity-dependent behavior of the DOR, which is also sequestered within DCVs in the spinal cord dorsal horn. The DOR redistributes the cell surface in a stimulation-dependent manner, binds more fluorescently labeled deltorphin after chronic inflammation, and stimulates greater agonist-dependent inhibition to intracellular cAMP after stimulation with the inflammatory mediator brandykinin. Because the DOR also colocalizes with substance P within DCVs, it is likely that there is a concurrent redistribution of these two receptors to the plasma membrane both of which would have (auto) inhibitory effects on the activity of the afferent.

Targeting of 5-HT_{1D} and DOR Receptors to DCVs

Given the remarkable similarities between the normal distribution of the DOR and the 5-HT_{1D} receptors, it is of interest to ask whether they may be cotrafficked from the DRG cell body to the terminal. Substance P interacts directly with the third luminal loop of the DOR, providing a mechanism for DOR trafficking to peptidergic terminals and an explanation for why DOR-IR is greatly reduced in the dorsal horn of mice lacking PPT-A, the gene that encodes the propeptide of substance P. Alignment of the putative binding domain of DOR with the corresponding region of the 5-HT_{1D} receptor (FIG. 7A) suggests that there is not an analogous interaction between the 5-HT_{1D} receptor and substance P, despite their likely colocalization within the same DCVs. A normal pattern of 5-HT_{1D}-IR in the dorsal horn of mice lacking the PPT-A gene is observed, suggesting that this particular mechanism of targeting DOR to DCVs does not represent a generalized process of sorting membrane proteins to this compartment. Moreover, because the 5-HT_{1D} receptor is dramatically reduced in the dorsal horn after peripheral nerve injury, the results also emphasize that the downregulation of the DOR by peripheral nerve injury is not necessarily a secondary consequence of the concurrent reduced expression of substance P. The mechanism for the concentration of 5-HT_{1D} receptor within DCVs and the subsequent regulation of this receptor at the plasma membrane remains to be determined.

Persistent Activation-Induced Changes in 5-HT_{1D}-IR

With persistent tissue injury (inflammation), a decrease in 5-HT_{1D}-IR was observed on day 3, a time when tissue swelling and the behavioral effects of CFA-induced hyperalgesia reach their peak. Prolonged nociceptor activation may underlie the relative depletion of the receptor from central terminals, leading to reduced negative feedback on the primary afferent, and enhanced nociceptive processing during this time. These findings parallel 5-HT_{1D} receptor gene expression levels, which declined slightly compared with the contralateral side. Although contralateral effects after inflammation are well known, their relative contribution is likely small compared with the ipsilateral changes in 5-HT_{1D}-IR under these conditions.

At day 7, when paw edema and the associated hyperalgesia are clearly normalizing, an increase in 5-HT_{1D}-IR in the ipsilateral dorsal horn is observed. The increase in receptor expression does not appear to reflect de novo expression of the receptor by afferents that normally do not express it. Rather, the distribution of 5-HT_{1D}-IR afferent terminals in the dorsal horn did not change, and DRG neurons with 5-HT_{1D}-IR continue to colocalize with substance P. Conceivably, the elevated levels of receptor provide greater negative inhibitory feedback on nociceptive afferents during the time of recovery from this type of tissue injury. This result also indicates that triptan administration, which targets the receptor, has an analgesic effect in these conditions.

Nerve Injury-Induced Depletion of 5-HT_{1D}-IR

Transection of the sciatic nerve induced a profound loss of 5-HT_{1D}-IR from the central terminals of the sciatic nerve. It follows that triptans are unlikely to exert a significant regulation of spinal cord nociceptive processing after this kind of nerve injury. The reduction in receptor levels at the nerve terminal is in marked contrast to the upregulation of 5-HT_{1D} gene expression in the DRG and the persistence of receptor in the cell body. The loss of 5-HT_{1D}-IR in the ipsilateral dorsal horn parallels the loss of DOR-IR after the nerve injury, and would thus also have the effect of amplifying pain in the setting of nerve injury.

### EXAMPLE 2

### Efficacy of Intrathecal Administration of Sumatriptan

The possibility of an analgesic action of sumatriptan on non-cranial pain, independent of the pain of headache, was examined. 5-HT_{1D} receptors are concentrated within dense core vesicles (DCVs) of the synaptic terminals. It was hypothesized that in the unstimulated state, sumatriptan lacks access to the sequestered receptor and thus should not influence acute pain. On the other hand, acute or chronic stimulation should trigger the redistribution of the receptor to the plasma membrane, making it available to activation by a triptan. To test this hypothesis functionally, the effects of systemic (subcutaneous; SC) or direct spinal (intrathecal; IT) injection of sumatriptan was studied in behavioral models of both acute and chronic pain. The results establish that appropriate targeting of triptans can in fact generate profound relief of pain other than that associated with migraine, including pain behaviors associated with tissue injury and inflammation. Furthermore, the spatial and temporal specificity of triptan analgesia suggests that the neurobiological mechanisms of triptan action depend upon the availability of the serotonin receptor subtype 1D (5HT1D) at the central terminals of sensory afferents in the spinal cord dorsal horn. Lastly, the dependence upon the intrathecal delivery of sumatriptan in reducing these experimental models of pain behavior in turn indicates that the underlying pathophysiology of migraine may involve a change in the blood brain barrier with respect to administered triptans. These results are relevant to the clinical use of triptans in a number of pain conditions, as well as to the understanding of the mechanisms of migraine pathophysiology.

Methods

Animals

Wild type CD1 male mice (20-30 g), housed in a 12-hour light-dark cycle, were used. Experiments were performed during the day by the same experimenter in a temperature and humidity controlled environment.

Administration of Drugs

Sumatriptan succinate, 12 mg/ml (GlaxoSmithKline) was diluted in preservative-free saline for injection in a suitable volume. For systemic administration, SC sumatriptan was given at 300 μg/kg and 600 μg/kg. For localized injection to the CNS, IT sumatriptan was given at 0.006 μg, 0.02 μg, 0.06 μg, and 0.6 μg in a total volume of 5 μl. These doses range from approximately 1/20th to 1/2000th the systemic dose. IT injections were performed with a 30 gauge, ½ inch needle at the L4-5 lumbar interspace on lightly restrained, unanesthetized mice (Fairbanks, 2003). Animals that exhibit motor impairments after the injection were excluded from study. In all nociceptive tests, mice were habituated to the test room and apparatus for 60 minutes on the day prior to the test and again immediately prior to the test.

Testing

Mechanical nociceptive thresholds were determined using a modification of the “up and down” method (Chaplan et al., J Neurosci Methods 1994; 53: 55-631994) with calibrated Semmes-Weinstein monofilaments North Coast Medical, Morgan Hill, Calif.). The starting filament was 3.61 (0.4 g), and the upper limit cutoff was 4.31 (2 g). To avoid further sensitization of animals with repeated testing, a lower limit cutoff was set in which four consecutive positive reactions with filaments of decreasing intensity would be scored as zero. Five animals were used in each treatment group. Thresholds were measured immediately prior to the administration of the test drug as well as at 30, 60, 90, 120, and 240 min. Acute thermal thresholds were measured with the hot plate test, set at 52.5° C. Response latency was defined as the time to the first nocifensive behavior, such as licking or jumping, with a cut off value of 50 sec. This test was performed 60, 120, and 240 min after administration of drug. Thermal hypersensitivity to carrageenan was measured by the withdrawal latency to focused radiant light using a PAW Thermal Stimulator (UC San Diego Department of Anesthesia), with a cut off value of 20 sec. Five animals were used in each treatment group. Paw withdrawal latencies were determined immediately prior to and 24 hours after carrageenan injection, and at 30, 60, 90, 120, 240 minutes after drug administration. The mean of three consecutive trials was recorded for each animal; the reported values represent the mean ±SEM of the group.

To screen for sedative and other adverse sensorimotor effects, mice were tested on a Rotarod (Ugo Basile, Comerio, Italy). The time in which mice were able to balance on a rod rotating on its axis at a constant velocity of 15 rpm were measured. The total duration of each trial was 300 sec. On the day prior to the test, animals accommodated to the task with three separate training trials. One hour prior to the test, the indicated dose and route of sumatriptan, saline or morphine was administered to five animals in each treatment group. A single trial was used for each dose and route; reported times represent % change from the baseline value for each animal ±SEM.

Models of persistent inflammation. For the carrageenan model, a 27-gauge needle was used to make an intradermal injection of 20 μl 3% carrageenan lambda (Sigma), dissolved in saline, in a lightly-restrained, awake animal. Hindpaw swelling pre- and 24 hours postinjection was used to confirm the effects of the injection. Five animals were used in each treatment group. For the formalin model, 10 μl of 2% formalin (Sigma) diluted in saline, was injected into the plantar surface of the left hind paw of a lightly-restrained, awake animal with a 27-gauge needle. Formalin induces biphasic pain behavior responses, divided into the phase 1 (0-10 minutes) and after interphase period with no pain behaviors, a phase 2 (10-60 minutes). Seven animals were used in each treatment group. The time spent licking and grooming the affected hindpaw, during both phases in 5-min bins, was measured. Animals received an injection of sumatriptan, morphine, or saline at the dose and route indicated one hour prior to the start of the formalin test.

Spared nerve injury (SNI) model of neuropathic pain. A model of partial sciatic nerve injury was used in which the peroneal and sural nerves were selectively ligated and cut, sparing the tibial nerve. Mice that did not develop mechanical allodynia on the fourth post-operative day (2 out of 12 animals) were excluded from the study. On postoperative days 7 and 8, mechanical thresholds were obtained immediately before and one hour after either IT saline or 0.06 μg IT sumatriptan. Animals were injected in a blinded cross-over manner, in which half of the animals received sumatriptan on one day and saline on the other.

Immunohistochemistry. For formalin stimulation, 5 mice were anesthesized with pentobarbital (60 mg/kg) and injected 80-100 μl of 5% formalin in phosphate buffered saline pH 7.4 into the plantar surface of the left hindpaw. After a terminal overdose of pentobarbital, the animals were perfused with saline at 5 min after hindpaw injection, followed by fixation with 10% formalin. For sciatic nerve cut, 5 mice were anesthetized with 2% isoflurane in oxygen, the sciatic nerve was exposed, ligated with 8-0 silk, and cut. As with SNI surgery, the surgery was closed in layers and skin closed with surgical clips. One week after surgery, animals were perfused as above following a terminal dose of pentobarbital. The lumbar L4 to L6 spinal cord was dissected, cut, stained, and analyzed, with the exception that the tissue was cut at 40 μm and every other section through this region stained. Sections were stained with an affinity purified rabbit anti-5HT_{1D} receptor antibody and detected by an ABC method with nickel-enhanced diaminobenzidine. Using the contralateral side as a control, the mean optical density of the medial portion of the dorsal horn from 3 sections of the lumbar segment was measured. The relative enhancement or loss of expression on the affected side is expressed as a ratio compared to the contralateral side.

Data presentation and statistical analysis. Data are represented as the means ±S.E.M. Mechanical and thermal threshold values were converted to the percentage of the maximum possible analgesic effect (% MPE), according to the formula % MPE=[(postdrug value−baseline value)/(cut-off value−baseline value)]×100. Statistical significance was assessed with ANOVA statistics, with correction for multiple comparisons in post-hoc analysis. A p value of <0.05 is considered significant and is indicated with an asterisk (*).

Intrathecal sumatriptan reduces the hyperalgesic effects of persistent inflammation induced by Complete Freunds Agent (CFA). CFA injection produces a prolonged period of mechanical hyperalgesia that persists and remains stable during days 3-7 after injection into the plantar surface of the hindpaw. CD1 male mice were used. Mechanical thresholds were tested on the first day, prior to a single injection of CFA to a hindpaw (baseline). By day 3 after CFA injection animals were hyperalgesic (post CFA 3d). Animals were tested for a sumatriptan response on 4 successive days (days 3-6) after CFA. A mechanical threshold was determined on each successive day prior to the administration of intrathecal sumatriptan. Doses of sumatriptan tested are: 0.0006 μg, 0.006 μg, 0.01 μg, and 0.06 μg, all in the volume of 5 μl. Mechanical thresholds were then determined at 30 and 60 min after the injection. The average withdrawal thresholds for the CFA-treated hindpaw at 30 and 60 mm after the administration of each dose of sumatriptan was measured. The antihyperalgesic effect of each of these doses, expressed as a percent of the pre-injection threshold for that day, were compared to the original baseline.

Persistent inflammation after CFA. For the CFA model, a 27-gauge needle was used to make an intradermal injection of 20 μl 50%. Complete Freund's Adjuvant (Sigma), emulsified in saline, in a lightly-restrained, awake animal. Hindpaw swelling pre- and 72 hours postinjection was measured to confirm the effects of the injection. Five animals were used in each treatment group.

Results

The effect of systemic injection of sumatriptan on acute thermal pain thresholds was tested. One test measured the latency of the reflex withdrawal of the hindpaw to a noxious heat stimulus applied to the hindpaw, and the second (the hot plate test) involved a more complex behavior that is presumed to result from integrated spinal and supraspinal “pain” transmission circuits.

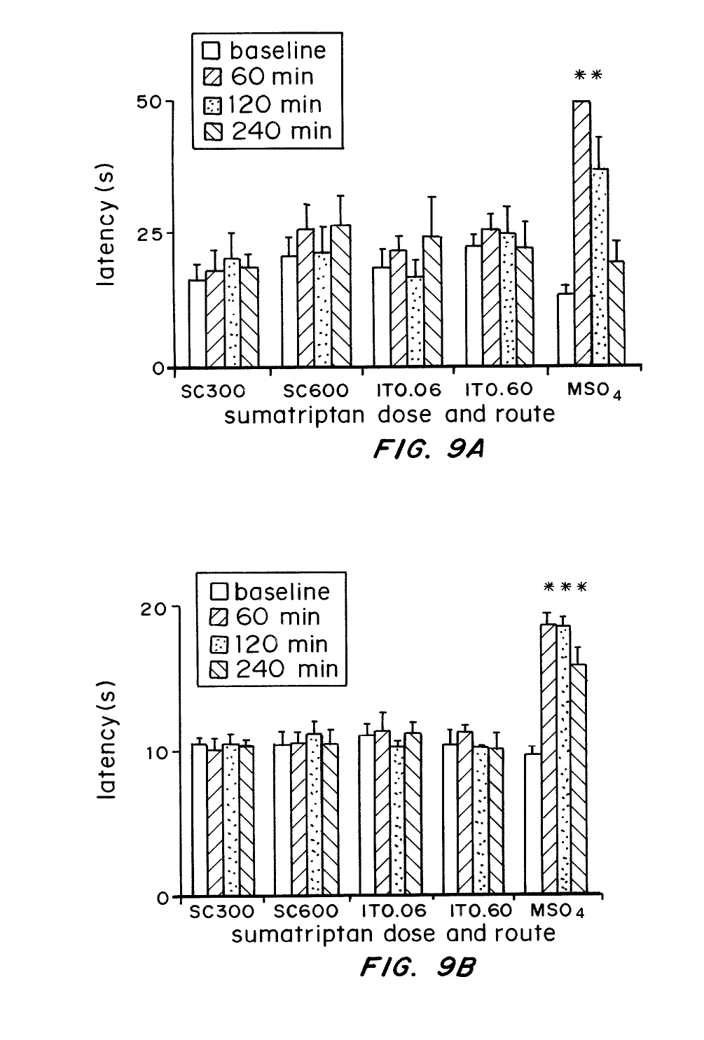

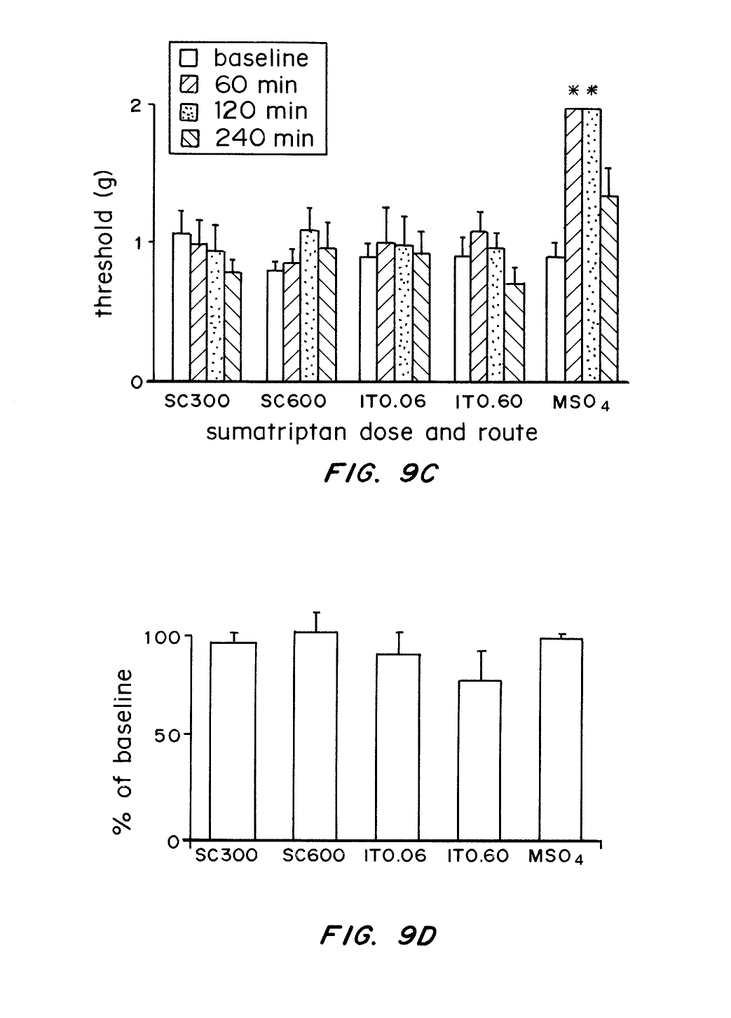

FIG. 9 shows that sumatriptan does not affect baseline pain thresholds. Tests of nociception by (A) hot-plate test, and (B) radiant heat to the hindpaw (Hargreaves test), or (C) mechanical pain using calibrated monofilaments, demonstrate no significant changes in threshold over the time course of the test after systemic (SC) or intrathecal (IT) administration of sumatriptan. SC doses were at 300 μg/kg (SC300) and 600 μg/kg (SC600), and IT doses were 0.06 μg (IT0.06) and 0.60 μg (IT0.60). A positive control of 10 nmol IT morphine sulfate (MSO₄) produced a robust analgesic response in these tests. These doses or routes of administration showed no evidence of confounding deficits due to sedation or sensorimotor incoordination on the Rotarod test.

Measured mechanical nociceptive withdrawal thresholds was measured with calibrated monofilaments. FIG. 9 illustrates that SC sumatriptan, at doses that inhibit neurogenic edema (i.e., regulate the release of transmitter from the peripheral terminals of nociceptors, had no effect on acute pain behaviors. Because sumatriptan is thought to cross the blood brain barrier (BBB) inefficiently, the effects of direct IT injections at 1/20th to 1/200th the systemic dose was measured. When administered by the IT route, it was found that sumatriptan was still completely without effect in these tests of acute pain. By comparison, these tests of acute pain are very responsive to morphine.

Sumatriptan Reduces Tissue Injury Pain

A model of persistent pain that triggers a massive exocytosis of DCVs, which would externalize 5-HT_{1D} receptors to the plasma membrane, and make them available for interaction with sumatriptan, was used. The formalin test is ideal for this analysis as it consists of two transient and stereotyped phases of pain behavior: the first phase is comparable to acute pain and is thought to result from direct activation of nociceptors; the second phase is a delayed inflammatory state, analogous to postoperative pain, which depends not only upon prolonged activity of nociceptors but also upon a first phase-induced central sensitization of pain transmission circuits within the spinal cord (Abbadie et al., 1997).

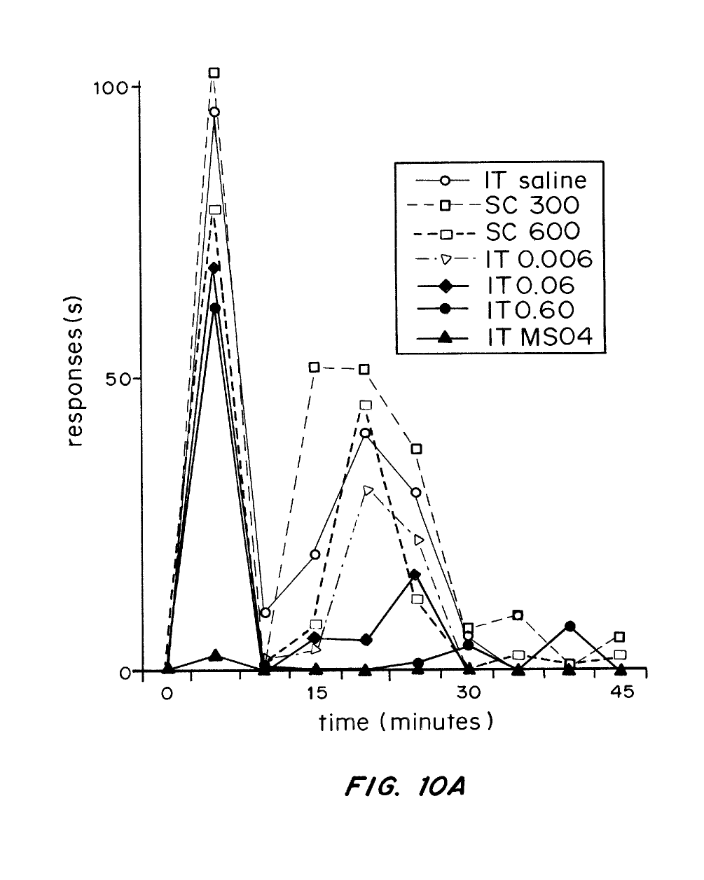

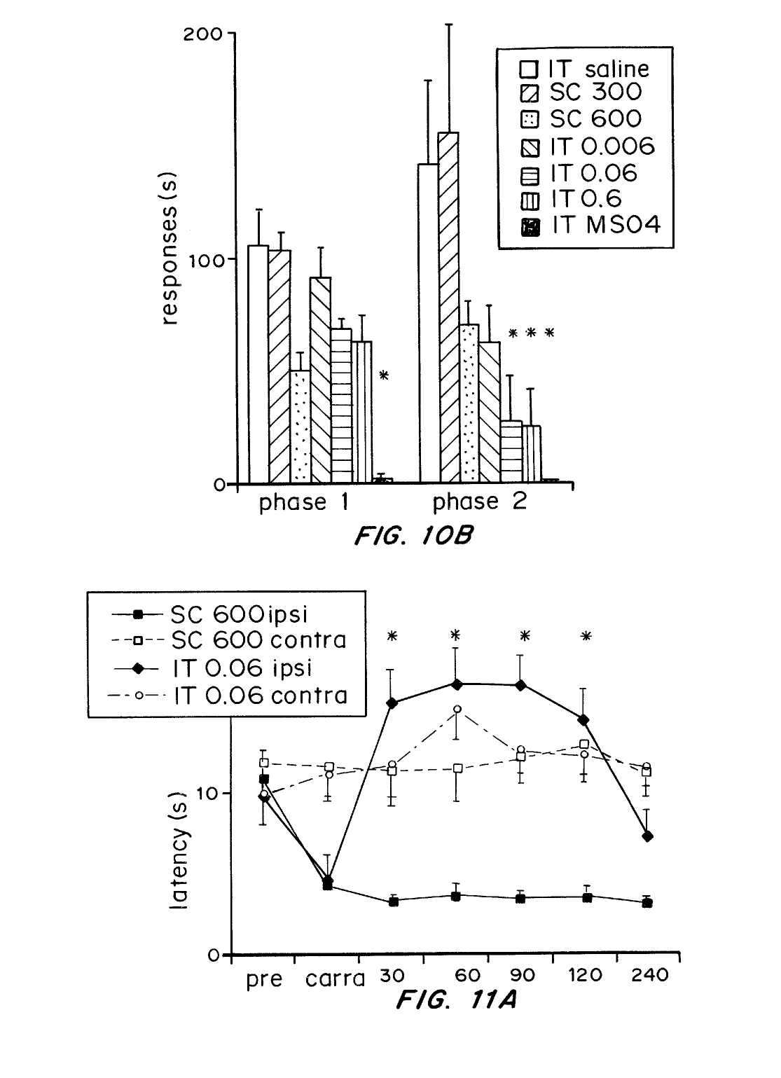

FIG. 10 illustrates that IT sumatriptan produced a profound reduction of pain behavior (analgesia) in the second phase of the formalin test. The formalin test began one hour after the administration of saline, sumatriptan, or morphine. The time course of hindpaw licking in 5 min bins (A), and the cumulative time spent licking in phase 1 (0-10 min) and phase 2 (11-60 min) (B) show that both IT saline- and SC sumatriptan-injected animals displayed stereotypical biphasic behaviors, but that only intrathecal (IT) administration of sumatriptan selectively and dose-dependently reduced the amount of second phase behaviors. SC doses of sumatriptan were 300 μg/kg (SC300) and 600 μg/kg (SC600). IT doses of sumatriptan were 0.006 μg (IT0.006), 0.06 μg (IT0.06) and 0.60 μg (IT0.60). A positive control of 10 nmol IT morphine sulfate (MSO₄) produced a robust analgesic response in this test.

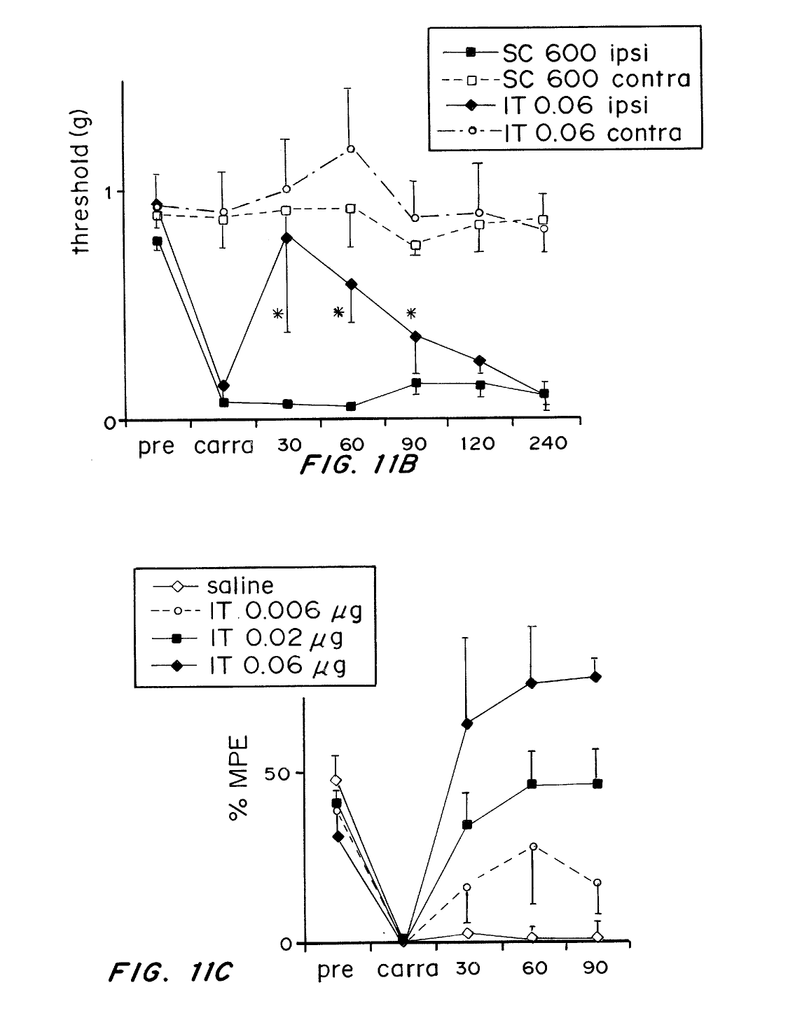

Systemic administration of sumatriptan modestly reduced second phase behaviors, but only at the highest dose tested, presumably because of limited spinal cord/brain penetration at these doses. The utility of sumatriptan in a model of hypersensitivity associated with tissue injury and inflammation, in which innocuous stimuli evoke pain behaviors (allodynia), was tested. Intradermal carageenan is an ideal model for these experiments, as it produces local inflammation and a pronounced thermal and mechanical hypersensitivity, within one hour of its injection. FIG. 11 shows that intrathecal, but not systemic sumatriptan, completely reversed the thermal and mechanical hypersensitivity in this model of persistent pain. The time-course of sumatriptan responses after sensitization by carageenan is shown to thermal (top) and mechanical (bottom) stimulation. The pre-test baseline is shown at left (pre). Thermal (A) and mechanical (B) thresholds are greatly reduced at 24 hours after injection of carrageenan to the left hindpaw (carra). Responses are shown over a time course (from 30 to 240 min) for a range of doses after the administration of or IT sumatriptan. Responses of the contralateral hindpaw (contra) remained unchanged throughout the procedure. Reduction of thermal (C) and mechanical (D) hyperalgesia by IT sumatriptan is dose-dependent, shown at 30-90 min after administration of drug (doses are as indicated in FIGS. 1 and 2). Values are given as percent of the maximal possible effect (% MPE).

The antinociceptive effect was significant 30 min after injection of sumatriptan, lasted for approximately one hour, and was dose-dependent. The behavior recorded after control injections of IT saline did not differ from that following SC sumatriptan. As expected, IT morphine produced a profound analgesia, with all animals reaching the cutoff latency. In a more recent analysis, IT sumatriptan also reduced mechanical hypersensitivity on days 3-7 after intraplantar injection of CFA.

Sumatriptan does not Influence Nerve Injury-Induced Pain

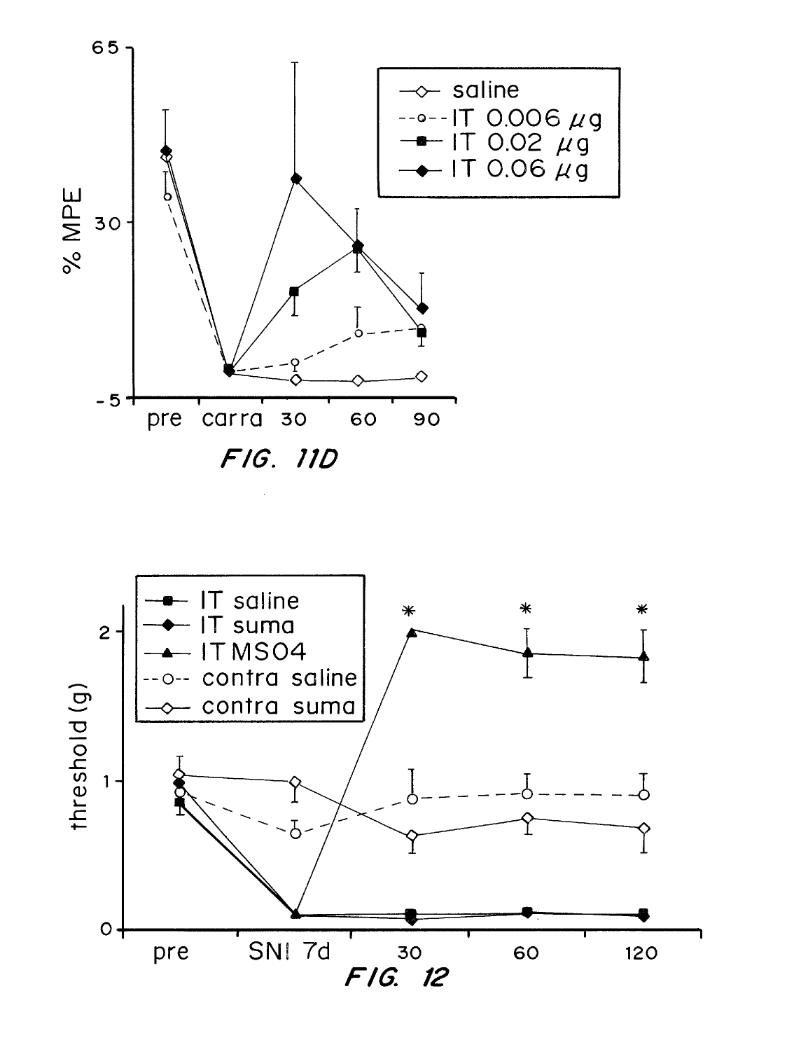

Because the pathophysiological mechanisms that underlie nerve injury-induced hyperalgesia involve changes in primary afferents and spinal cord dorsal horn that are distinct from those of chronic inflammation, an experimental form of nerve injury that models a neuropathic pain condition in patients was used. In this model of nerve injury pain, two of the three branches of the sciatic nerve were transected, sparing the tibial branch, which permits behavioral testing of the plantar surface of the hindpaw. Mice demonstrate a pronounced mechanical hypersensitivity of the partially denervated hindpaw, within two days of the denervaion. In contrast to the profound analgesic action of sumatriptan for inflammatory pain, sumatriptan was completely without effect on the mechanical hypersensitivity produced by nerve injury, regardless of dose or route of delivery. As expected, IT morphine produced a profound analgesia, to cutoff latencies (FIG. 12A). Spared nerve injury of the sciatic nerve establishes a mechanical hyperalgesia ipsilateral to the injury that is stable and fully developed at 7 days post-nerve transection (SNI 7d). Neither IT sumatriptan 0.6 μg (IT suma) nor IT saline (IT saline) reduced SNI-induced hypersensitivity, 30-120 min after administration of drug, whereas IT morphine (IT morphine) produced a significant analgesia. Nociceptive thresholds of the unaffected contralateral leg (contra) are unaffected by the treatment of saline or sumatriptan. (B) Tissue from lumbar spinal cord, taken 5 min after the injection of dilute formalin into the hindpaw, shows a marked increase in 5-HT_{1D}-IR in the dorsal horn of the spinal cord on the side of the injection. The increase is most pronounced in the medial half of the dorsal horn, which receives afferent input from the hindpaw. (C) In a mouse studied 7 days after transection of the sciatic nerve, there is a depletion of 5-HT_{1D}-IR in the spinal cord ipsilateral to the lesion, which matches the timing of the behavioral testing after SNI. As predicted by our model of receptor availability, there was no effect of sumatriptan on first phase pain behavior, which is comparable to acute pain. However, IT sumatriptan reduced pain behaviors in the second phase of the formalin test in a dose dependent manner. In contrast to sumatriptan, morphine eliminated both first and second phase behaviors.

Regulation of 5-HT_{1D} Receptor Distribution after Tissue and Nerve Injury.

Analysis of the dorsal horn distribution of the 5HT_{1D} receptor in mice after tissue or nerve injury provided a likely explanation for the differential responsiveness of inflammatory and neuropathic pain behaviors to sumatriptan. To bridge the possible differences in 5HT_{1D} receptor behavior here with the previous anatomic experiments in rat, it was shown that acute inflammatory and nerve injury models used in these mouse behavioral models also initiate corresponding changes in 5HT_{1D} receptor expression. FIG. 12B illustrates that there is an increase in 5-HT_{1D} receptor immunoreactivity (5-HT_{1D}-IR) in the ipsilateral spinal cord dorsal horn five minutes after injection of formalin into the hindpaw. This time point corresponds both to the most intense period of nociceptive behaviors in the formalin test as well as to the period of injury-evoked discharge of primary afferents. In fact, the time frame corresponds roughly to the time it takes to detect the release of peptide neurotransmitters from the DCVs that sequester the receptor. As was the case after CFA injection, it was found that the receptor expression pattern after persistent inflammatory injury does not correlate well with the behavioral manifestations of hyperalgesia. Specifically, the pattern of 5HT_{1D}-IR at one day after carrageenan injection did not differ from that of the contralateral side. By contrast, a significant decrease of 5-HT_{1D}-IR in the dorsal horn ipsilateral to the peripheral nerve injury was observed. Thus the failure of sumatriptan to modulate neuropathic pain likely reflects downregulation of this receptor at the central terminal of nociceptors. The extent of 5HT_{1D}-IR loss at one week after the injury corresponds to the timing of the behavioral experiment after spared nerve injury in FIG. 12.

Summary

Sumatriptan can reduce the pain of inflammation in non-migrainous, non-cranial regions of the body, when given intrathecally. Systemic administration of sumatriptan was without effect even at doses 200-fold greater than the effective intrathecal dose, demonstrating the potent analgesic effect of sumatriptan in models of tissue injury pain when administered intrathecally. In the unstimulated baseline state, even intrathecal sumatriptan was entirely ineffective against acute thermal or mechanical pain thresholds (FIG. 9), establishing that the failure of systemic sumatriptan to reduce acute pain was not merely due to its limited ability to cross the BBB. In contrast to acute pain, intrathecal sumatriptan produced a selective and profound inhibition of the second but not the first phase of the formalin test (FIG. 10), as well as the hypersensitivity associated with tissue inflammation (FIG. 11). Intrathecal sumatriptan not only completely reversed thermal hyperalgesia but also revealed an analgesic effect (i.e. latencies exceeded those at baseline). Also, despite the dramatic and complete reversal of hypersensitivity of the carrageenan-treated hindpaw, sumatriptan did not affect pain thresholds in the unstimulated contralateral hindlimb. This localized action of sumatriptan to the area of tissue injury is consistent with the functional availability of receptors only in afferents stimulated by noxious inputs. The fact that sumatriptan only influenced pain behavior generated by nociceptors that were sensitized after prior injury, taken together with the requirement of an intrathecal route of administration, argues strongly that the central terminal of the primary afferent nociceptor is a major target of sumatriptan for the relief of inflammatory pain and that 5HT_{1D} receptors are a critical target for pain control, as indeed they specify the conditions under which the receptor is accessible to a triptan. The greater efficacy of intrathecal over systemic sumatriptan in reversing inflammation induced pain emphasizes that the BBB is, in fact, a critical factor in triptan action.

Those skilled in the art will recognize, or be able to ascertain using no more than routine experimentation, many equivalents to the specific embodiments of the invention described herein. Such equivalents are intended to be encompassed by the following claims.

We claim:

1. A dosage unit formulation for the intrathecal treatment of pain, comprising a therapeutically effective amount of a triptan to treat non-migrainous tissue pain when administered intrathecally, in combination with a pharmaceutically acceptable carrier for intrathecal administration.

2. The formulation of claim 1, wherein the triptan is selected from the group consisting of rizatriptan, eletriptan, naratriptan, zolmitriptan, frovatriptan, sumatriptan, almotriptan, and combinations thereof.

3. The formulation of claim 1 further comprising a second agent for pain control.

4. The formulation of claim 3 wherein the second agent is selected from the group consisting of morphine, clonidine, fentanyl and baclofen.

5. A method of treating pain, comprising administering intrathecally to a patient in need thereof an effective amount of a triptan to treat non-migrainous tissue pain when administered intrathecally, in combination with a pharmaceutically acceptable carrier for intrathecal administration.

6. The method of claim 5 wherein the triptan is selected from the group consisting of rizatriptan, eletriptan, naratriptan, zolmitriptan, frovatriptan, sumatriptan, almotriptan, and combinations thereof.

7. The method of claim 5 comprising administering the triptan in combination with a second agent for pain control.

8. The method of claim 7 wherein the second agent is selected from the group consisting of an opiate, clonidine, fentanyl and baclofen.

9. The method of claim 5 comprising administering the triptan in combination with gabapentin or pregabalin.

10. The method of claim 5 wherein the triptan is administered to a patient for the treatment of a condition selected from the group consisting of cancer pain, chronic back pain, rheumatoid arthritis, osteoarthitis, post-herpetic neuralgia, and complex regional pain syndrome types I or II, posttraumatic or post-operative pain, diabetic vasculopathy, inflammatory radiculopathy, and inflammatory plexopathies such as brachial plexopathy (Parsonage Turner syndrome) or lumbar plexopathy.

11. The method of claim 5 wherein the patient has neuropathic pain in humans.

12. The method of claim 11 wherein the triptan is administered to a patient for the treatment of a condition selected from the group consisting of HIV neuropathy, chemotherapy-induced neuropathy (such as vincristine toxicity), erythromelalgia, diabetic neuropathy, and inherited painful disorders such as metachromatic leukodystrophy, Friedreich's ataxia, and Fabry's disease.

13. The method of claim 5 wherein the triptan is administered for acute pain management.

14. The method of claim 5 for treatment of pain secondary to spinal cord injury.

15. The method of claim 5 wherein the triptan is administered for labor management or spinal blockade for surgery.
